(19) United States
(12) Patent Application Publication
(10) Pub. No.: US 2025/0170381 A1
(43) Pub. Date: May 29, 2025

(54) PORT

(71) Applicant: NEUROCHASE TECHNOLOGIES LIMITED, Bristol (GB)

(72) Inventor: Steven Streatfield GILL, Bristol (GB)

(73) Assignee: NEUROCHASE TECHNOLOGIES LIMITED, Bristol (GB)

(21) Appl. No.: 18/841,966

(22) PCT Filed: Feb. 22, 2023
(86) PCT No.: PCT/GB2023/050401

§ 371 Date: Aug. 27, 2024

(30) Foreign Application Priority Data
Feb. 28, 2022 (GB) 2202719.7

(51) Int. Cl. A61M 39/02

(52) U.S. Cl. CPC A61M 39/0247; A61M 2039/0223; A61M 2039/025; A61M 2039/0261; A61M 2039/0264; A61M 2039/027; A61M 2039/0276; A61M 2039/0282; A61M 2039/0288; A61M 2202/0007; A61M 2202/0014; A61M 2202/04; A61M 2205/0205; A61M 2205/0216; A61M 2205/0233; A61M 2205/0255; A61M 2205/0261; A61M 2210/0687

(57) ABSTRACT

A device for providing fluid access to a mammals central nervous system comprising: a fluid port allowing the fluid access; a housing comprising an extracorporeal portion and a lowermost surface to engage with the skull; and a fluid tube connected to the fluid port and extending below the housing. In various aspects: no part of the housing extends below the lowermost surface and the fluid tube bends to run along a trench in the skull; the lowermost surface comprises teeth; a septum seals the fluid port and a cap engages with the extracorporeal portion to compress the septum; and the device comprises a guide member and a connector having needles for fluid connection via the fluid port engages with the guide member, where, upon engagement of the connector and guide member, the needles adopt predetermined positions. Also provided is a method of implanting the device.

The present invention relates to a medical device for providing fluid access for delivery or removal of fluids from the body. In particular, it relates to a device for providing fluid access to the central nervous system of a mammal.

The direct administration of therapeutic agents to the central nervous system (CNS) has been investigated for many years with the aim of bypassing the blood-brain barrier (BBB) and minimising the risk of off-target and systemic side effects from the therapeutic agents.

Direct drug delivery to the CNS dates back to 1885 with the first lumbar puncture to administer cocaine for anaesthesia (Corning). The introduction of therapies directly into the cerebrospinal fluid (CSF) via intraventricular or intrathecal injections or infusions has continued to evolve and includes the use of implantable pumps for chronic infusions. These methods of administration are used for the treatment of a variety of conditions and disorders including pain, spasticity, leptomeningeal carcinomatosis, and microbial infections. Experimentally, administration for the treatment of neurodegenerative diseases such as Parkinson's disease, Alzheimer's disease, Huntington's disease, spinal muscular atrophy and lysosomal storage diseases has also been investigated.

Therapies have also been infused directly into targeted volumes of the brain parenchyma for the treatment of neurodegenerative diseases, lysosomal storage diseases and brain tumours. This can be achieved using the method of Convection Enhanced Delivery (CED) that was first described by Bobo in 1994. Here, an infusate of the therapy is delivered at a carefully controlled flow rate through a fine cannula placed in the brain target volume such that a pressure gradient is established at the cannula's port that drives the infusate into the tissue. The infusate carries the drug by bulk flow, displacing extracellular fluid and achieving a homogenous drug concentration. CED facilitates the accurate anatomical targeting and delivery of therapeutic drug concentrations through clinically relevant volumes of brain tissue or tumour.

Using such methods, the BBB can act to retain drugs within the brain and to reduce systemic side effects. A number of neurological diseases treatable by infusion into the CSF or by direct infusion into the brain parenchyma require repeated dosing over months or years. To avoid the patient having to undergo repeated implantation of cannulas for each treatment, cannulas can be left in-situ and connected to implantable infusion pumps. However, the use of implantable pumps is limited because infusion regimens can be complex for many therapies, and available programmable pumps cannot meet the requirements. One pump is required per cannula and intraparenchymal drug delivery using CED may require in excess of four implants which become cumbersome for patients as pumps are relatively bulky. The greatest contraindication to the use of implantable pumps is that many drugs will degrade when stored at body temperature in the pump reservoir. In addition, filling the reservoir transcutaneously poses a cumulative risk of infection, or in some cases inducing an immune response to a proteinaceous therapy from repeated subcutaneous inoculation.

An alternative to the deployment of implantable pumps for intermittent infusions of therapy to the CNS is to provide an implantable, septum-sealed reservoir that is connected to a cannula which can be accessed transcutaneously. An example is described in EP 1426074 B1.

The shortcomings of such devices for repeated infusions to the CNS are the requirement to penetrate the skin to gain access to the reservoir. This carries a cumulative risk of infection or inducing an immune response to the therapy. Retaining transcutaneous needles in a subcutaneous reservoir for long periods during infusions increases these risks, and also increases the risk of needle displacement. Additionally, if multiple cannulas need to be deployed, equal numbers of reservoirs will need to be implanted. This proportionately increases the risks.

Various attempts have been made to provide improved devices for such purposes.

WO 2007/104961 describes a subcutaneously-implantable, septum-sealed fluid connector having a plurality of lumens for connecting to cannulas. The cannulas can be connected to infusion pumps when required by cutting down to the connector and attaching it to a male counterpart with a plurality of needles, each connected to an infusion line and pump. The disadvantage of this system is the requirement for the patient to undergo repeated surgeries to open and close a wound to access the connector. Apart from the associated inconvenience and discomfort there is an increased risk of infection.

WO 2008/062173A describes a percutaneous access device for neurological applications that permits repeated CED infusions of therapies into the CNS, thereby avoiding repeated surgeries and the risks associated with multiple needle penetrations of the skin. This apparatus comprises at least one intracranial catheter connected to at least one port housed within the body of the device wherein the body has an extracorporeal surface and a subcutaneous surface. The lumen of the port can be accessed from the extracorporeal surface through a seal and the device is stabilised with a subcutaneous flange which is perforated to encourage bio-integration with the subcutaneous tissue. The shortcoming of this device is that in order for a hermetic seal to be created at the tissue/device interface to prevent infection tracking into the subcutaneous tissue and thence to the brain, the tissue needs to integrate into the surface of the device. Due to the inherent mobility of the skin and movement of the device in the soft tissues, such integration is very unlikely to occur. Therefore, such devices become marsupialised and infected, posing a significant risk to patients. U.S. Pat. No. 8,827,987 B2 describes a percutaneous bone-anchored, device for drug delivery to the CNS. The device comprises one or more ports for supplying fluid to one or more cannulas implanted in the brain that are accessible from the extracorporeal surface through a septum seal. The device is inserted into a complementary recess formed in the bone where it is retained by features that grip the internal surface of the recess. A number of problems are encountered when deploying such a device.

First, the creation of a recess in the bone to complement the device's profile requires the use of guided instruments to accurately machine the bone to facilitate an effective interference fit that will secure the device. This requires the use of image guided surgery and a stereoguide or robot to guide instruments to machine the skull to a known depth so as not to penetrate the brain. It may also require the placement and fixation of a jig to the skull thereby increasing the size of the wound required to implant the device. Hence implantation is complex and the procedure time is long, exposing patients to greater risk of infection and surgical complications.

Second, the skull may be thin especially in children, for example 2 mm thick, and in such circumstances there will be insufficient engagement of the device with the internal surface of the bone recess to retain it. Penetration of the full skull thickness also creates a direct path from the extracorporeal surface through the skin and bone to the meninges surrounding the brain so that unless a hermetic seal is created at the device/bone interface upon device implantation then there is a risk of infection with meningitis or extradural abscess. Implantation of the subcutaneous portion of the device through thin bone will also result in it compressing brain tissue.

Third, U.S. Pat. No. 8,827,987 B2 teaches that the device may be forced into tight engagement with the bone recess using an impactor or other tool to provide a friction fit, simplifying surgical implantation and creating a more reliable attachment of the device to a subject than can be achieved using glue, screws or the like. The converse may however be the case because abnormally high stress concentrations in the bone from impaction can result in pressure necrosis and subsequently in the loosening and failure of the implant.

WO97/49438 describes a transcutaneous fluid transfer apparatus comprising a plate that can be fixed to the skull using bone screws. WO99/34754 also discloses a percutaneous transferring device that can be screwed to bone.

In view of the shortcomings of the above-described prior art devices, there is still a need for improved devices for providing fluid access to the central nervous system of a mammal.

According to a first aspect of the invention, there is provided a device for providing fluid access to the central nervous system of a mammal, said device comprising: a fluid port allowing the delivery or removal of fluid from the central nervous system; a housing comprising an extracorporeal portion allowing access to said fluid port, and a lowermost surface configured to engage with an outermost surface of the skull; and a fluid tube connected to the fluid port and extending below the housing through the lowermost surface, wherein: no part of the housing extends below the lowermost surface; and the fluid tube is configured to bend so as to run along a trench formed in the outermost surface of the skull.

Arranging the device such that no part of the housing extends below the lowermost surface that engages the skull removes the need to cut a precisely-sized recess in the skull to accommodate the device. This greatly reduces the time required for implantation of the device, and the need for additional jigs or guides to be screwed to the skull as in prior art devices. Thereby the risk to the patient from extended surgery time and large wound sizes is reduced. The lack of a need for a precise fit between the device and a recess in the skull also reduces the risk of infection, pressure necrosis, or other risks associated with prior art devices.

Optionally, no part of the device extends through an interior surface of the skull when the lowermost surface of the housing is engaged with the outermost surface of the skull. Avoiding breaching the interior surface of the skull greatly reduces the risk of infection entering the brain of the patient.

Optionally, the lowermost surface is configured to engage with, and attach to, the outermost surface of the skull using a plurality of screws. Screws provide a rigid connection to the skull that prevents relative movement of the device and skull. This reduces the risk of marsupialisation around the device.

Optionally, the fluid tube is configured to bend at a point below the lowermost surface. This allows the fluid tube to run along and inside the trench starting from a region protected by the covering of the device itself. This reduces exposure of the fluid tube to the external environment, and the corresponding risk of infection where the tube enters the skull.

According to a further aspect of the invention, there is provided a device for providing fluid access to the central nervous system of a mammal, said device comprising: a fluid port allowing the delivery or removal of fluid from the central nervous system; a housing comprising an extracorporeal portion allowing access to said fluid port, and a lowermost surface configured to engage with an outermost surface of the skull; and a fluid tube connected to the fluid port and extending from the housing, wherein: the lowermost surface of the housing comprises a plurality of teeth for engagement with the outermost surface of the skull.

Providing a plurality of teeth of the lowermost surface that engages the skull reduces the risk of relative movement of the device and skull. This reduces the risk of marsupialisation around the device. The teeth may also contribute to displacing cement used to seal the device to the skull, so as to better fill voids between the device and the skull surface that would otherwise increase risk of infection.

Optionally, the plurality of teeth is distributed over the lowermost surface. Optionally, the plurality of teeth is distributed over at least 50% of an area of the lowermost surface. Distributing the teeth over the surface ensures a uniformly secure engagement of the device, further reducing the risk of relative movement.

According to a further aspect of the invention, there is provided a device for providing fluid access to the central nervous system of a mammal, said device comprising: a fluid port allowing the delivery or removal of fluid from the central nervous system; a housing comprising an extracorporeal portion allowing access to said fluid port; a fluid tube connected to the fluid port and extending from the housing; a septum sealing the fluid port; and a cap configured to engage with the extracorporeal portion of the housing and compress the septum when the cap is attached.

A cap is desirable to protect the septum from mechanical damage and the effects of ultraviolet light, which can degrade materials such as silicone that may be used for the septum. The cap also ensures cleanliness of the septum when the fluid port is not in use. The cap prevents debris such as dirt, infected material, grease, hair, or skin from becoming ingrained in the septum, which could then be driven into the brain via the fluid port when the device is in use. By providing a cap that compresses the septum when not in use, the fluid port can be effectively sealed with a thinner septum than in prior art devices. When the cap is removed so the septum can be pierced with a needle for fluid transfer, the relatively thin and un-compressed septum is less likely to be cored by the passage of the needle. There will also be a significant reduction in shear forces imposed on the septum by the passage of a needle, thereby reducing the formation of wear debris and the degradation of the septum that can compromise its sealing effect. Wear debris is undesirable, as it can block the fluid tube or be carried into the central nervous system where it may provoke inflammation.

Optionally, the cap is configured to compress the septum by applying a force perpendicular to a plane of the septum. This means that the fluid port can be effectively sealed without applying radial forces that are more likely to make needle insertion difficult and lead to coring of the septum.

Optionally, the cap is configured to engage with the extracorporeal portion using a mechanical connection. A mechanical connection provides a secure and reversible method of attaching the cap.

Optionally, the mechanical connection comprises a first connection feature on the cap and a second connection feature on the extracorporeal portion, and the cap is configured to engage with the extracorporeal portion by engagement of the first connection feature with the second connection feature. Engagement of opposing features on both cap and housing can provide a more secure engagement of the cap with the housing, and permit a more consistent application of the compression force.

Optionally, the mechanical connection is configured such that a predetermined compression force is applied to the septum when the cap is engaged with the extracorporeal portion. By ensuring a predetermined force is applied, it can be ensured that the force is sufficient to compress the septum and seal the port, while not being so large as to risk damage to the septum or other parts of the device.

Optionally, the cap is configured to provide a seal around the septum. This further assists in ensuring cleanliness of the surface of the septum, and reducing the risk of dirt, debris, or pathogens being introduced into the CNS via the fluid port.

Optionally, the device further comprises a connector cap configured to engage with the extracorporeal portion of the housing, the connector cap comprising a needle configured to make fluid connection with the fluid tube via the fluid port. The connector cap is a convenient way to connect the device to a reservoir or other external source of fluids that are to be administered to the central nervous system.

Optionally, the connector cap is configured to engage with the extracorporeal portion using a mechanical connection. A mechanical connection provides a secure and reversible method of attaching the cap.

Optionally, the mechanical connection comprises a first connection feature on the connector cap and a second connection feature on the extracorporeal portion, and the connector cap is configured to engage with the extracorporeal portion by engagement of the first connection feature with the second connection feature. Engagement of opposing features on both cap and housing can provide a more secure engagement of the cap with the housing, and permit a more consistent application of the compression force.

Optionally, the device comprises a septum sealing the fluid port, and the connector cap is configured to compress the septum by applying a force perpendicular to a plane of the septum when the connector cap is engaged with the extracorporeal portion of the housing. This means that the septum can provide an effective seal around the needle without applying radial forces that are more likely to make needle insertion difficult and lead to coring of the septum.

Optionally, the mechanical connection is configured such that, upon engagement of the connector cap with the extracorporeal portion, the needle advances a predetermined distance through the septum. Configuring the connector cap in this way ensures that the needle is advanced a sufficient distance to make a reliable fluid connection between the needle and the fluid port of the device, without risking damage to any components if the needle is advanced too far.

Optionally, the connector cap further comprises: a second fluid tube in fluid connection with the needle and extending from a side of the connector cap opposite to the needle; and a plurality of grooves configured to retain the second fluid tube. The second fluid tube allows the connector cap to be connected to a reservoir or a syringe pump used to administer a therapeutic agent, and the grooves in the connector cap allow the fluid tube to be retained in a convenient position during use of the device, thereby reducing the risk of misadministration or damage to any components during delivery of fluid.

According to a further aspect of the invention, there is provided a device for providing fluid access to the central nervous system of a mammal, said device comprising: a fluid port allowing the delivery or removal of fluid from the central nervous system; a housing comprising an extracorporeal portion allowing access to said fluid port; a guide member; one or more fluid tubes connected to the fluid port and extending from the housing; and a connector configured to engage with the guide member, the connector comprising one or more needles configured to make fluid connection with respective ones of the fluid tubes via the fluid port, wherein the connector and the guide member are configured such that, upon engagement of the connector with the guide member, each of the needles adopts a predetermined position relative to the respective one of the fluid tubes.

The use of a connector and guide member greatly simplifies the operation of the device by removing the need for a user to align the needle with the fluid port by eye. This is particularly advantageous if plural fluid ports and needles are provided for delivery of different therapies, because the risk of administering a therapy via an incorrect fluid port is greatly reduced. The guide member also ensures that the needle is always guided through the fluid port at the same position. Particularly where a septum is used to seal the port, this can reduce wear on the seal and improve longevity of the device.

Optionally, the device comprises a septum sealing the fluid port; and the connector and the guide member are configured such that, upon engagement of the connector with the guide member, each of the needles advances a predetermined distance through the septum. This ensures that the needle is advanced sufficiently far to penetrate the septum and reach the fluid port, but not so far as to risk damage to the needle or other components of the device such as the fluid tube.

Optionally, the guide member comprises a plurality of guide posts; the connector comprises a cam configured to engage with the guide posts; and the cam and guide posts are configured such that rotation of the cam causes the advancement of the needle by the predetermined distance to the predetermined position. The use of guide posts and a cam arrangement ensures that an appropriate level of force can be applied in a controlled manner to advance the needle through the septum. By utilising the mechanical advantage provided by the cam, the cam and guide member also reduce the force that must be applied to the device to advance the needle, providing mechanical advantage for the user. This improves reliability of fluid connection to the fluid port while also reducing the likelihood of damage to the device during use. Further, the guide posts can be designed to have a large aspect ratio of length to diameter, which reduces the angular deviation possible when engaging the connector. Using multiple, smaller diameter guide posts provides more precise guidance over a shorter distance compared to prior art designs where a relatively short, wide cylinder is engaged with a recess in the skull. This allows a low-profile connector to be used, reducing the likelihood of the device being knocked and causing harm in clinical use.

Optionally, the cam and guide posts are further configured to reversibly lock the needle at the predetermined position once the needle has advanced by the predetermined distance. Removably locking the needle greatly reduces the chance of the needle moving during use of the device, further improving the reliability of the fluid connection to the fluid port.

Optionally, the guide member is removably attached to the extracorporeal portion of the housing using a mechanical connection. A removable guide member reduces the size of the device when not in use, providing improved convenience for the user. It also allows for different guide members to be provided depending on the configuration of the device and connector.

Optionally, the mechanical connection comprises a first connection feature on the guide member and a second connection feature on the extracorporeal portion, and the guide member is removably attached to the extracorporeal portion by engagement of the first connection feature with the second connection feature. Engagement of opposing features on both cap and housing can provide a more secure engagement of the cap with the housing, and permit a more consistent application of the compression force.

Optionally, the mechanical connection comprises one or more of a thread, a snap fit connection, an interference fit connection, and a grub screw. These mechanical connections can provide sufficient retaining force, while also being made sufficiently small and easy to use to be appropriate for use in the device.

Optionally, a surface of the housing configured to be in contact with tissue of the mammal has at least one of a texture and coating configured to promote tissue integration. Promoting tissue integration with the device reduces the risk of bacterial ingress that can cause inflammation and infection.

Optionally, the housing of the device comprises titanium and/or polyetheretherketone. Titanium has high strength and relatively low density, while also being biocompatible. It therefore makes a good material for medical implants. PEEK is also biocompatible and lightweight.

Optionally, the device further comprises a septum sealing the fluid port, wherein the septum is a pre-pierced or split septum. A pre-pierced or split septum allows a needle to pass through the septum while reducing the risk of creating debris as the needle punctures the septum. This reduces the chance of introducing unintended foreign matter into the central nervous system.

Optionally, the housing comprises one or more protrusions configured to compress the septum. This allows the fluid port to be effectively sealed with a thinner septum than in prior art devices, by compressing the septum using features on the housing, similarly as discussed above for the cap.

According to a further aspect of the invention, there is provided a kit for implanting a device for providing fluid access to the central nervous system of a mammal, the kit comprising: the device of any preceding claim; and a predetermined quantity of an acrylic cement.

Acrylic cement is particularly suitable for aiding in fixation of the device to the skull, since it can fill gaps and irregularities around the device and any incisions into the skull made for the purpose of fixing the device. This prevents ingress of foreign materials or contaminants that could cause infection or inflammation. However, acrylic cement suitable for medical applications is typically provided in relatively large quantities and cures rapidly. Providing a predetermined quantity of cement as part of a kit with the device ensures that an appropriate quantity of cement is available when implanting the device, and reduces wastage of unused cement.

Optionally, the acrylic cement comprises an antimicrobial agent. Incorporating an antimicrobial agent further reduces the risk of infection following implantation of the device.

According to a further aspect of the invention, there is provided a method of implanting a device for providing fluid access to the central nervous system of a mammal, the method comprising: forming a trench in the outermost surface of the mammal's skull, said trench extending from an implantation site towards a cannula providing fluid connection to the central nervous system of the mammal, wherein the trench does not penetrate the inner surface of the skull; connecting a fluid tube of the device to the cannula; filling the trench with an acrylic cement; inserting the fluid tube into the trench; and engaging the lowermost surface of the device with the outermost surface of the skull at the implantation site.

Forming a trench and inserting the fluid tube into the trench provides a method of implanting the device that can be carried out quickly and requires less precision than existing methods of implanting similar devices, which require guided instruments and jigs to create shaped recesses in the skull matching the profile of the device being inserted. This means that surgical invasiveness and length of surgery are both reduced, greatly decreasing the risk of the procedure to the patient. Filling the trench with an acrylic cement retains the tube in the trench and prevents infection by filling voids around the tube and the device.

Optionally, the method further comprises removing an area of scalp at the implantation site of sufficient size to accommodate an extracorporeal portion of a housing of the device. Removing an area of scalp allows the skin to fit around the device and aid its integration.

Optionally, the method further comprises removing subcutaneous fat and hair follicles in a predetermined area around the implantation site. Removing the relatively mobile subcutaneous fat between the skin's dermis and the periosteum results in their fusion, so that when the combined layers engage with the surface of the device the reduced mobility of the dermal layer encourages its integration with the device. This lowers the risk of marsupialisation and infection as well as allowing the skin and periosteum immediately over the subcutaneous portion of the device to lay more level with the surrounding skin.

Embodiments of the present invention will now be described by way of non-limitative example with reference to the accompanying drawings, in which:

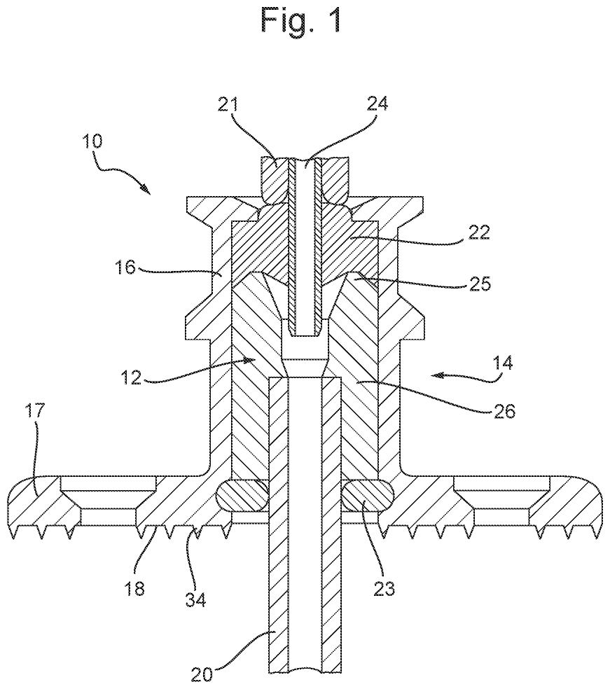

FIG. 1 is a cross-sectional view of a device having a single fluid port;

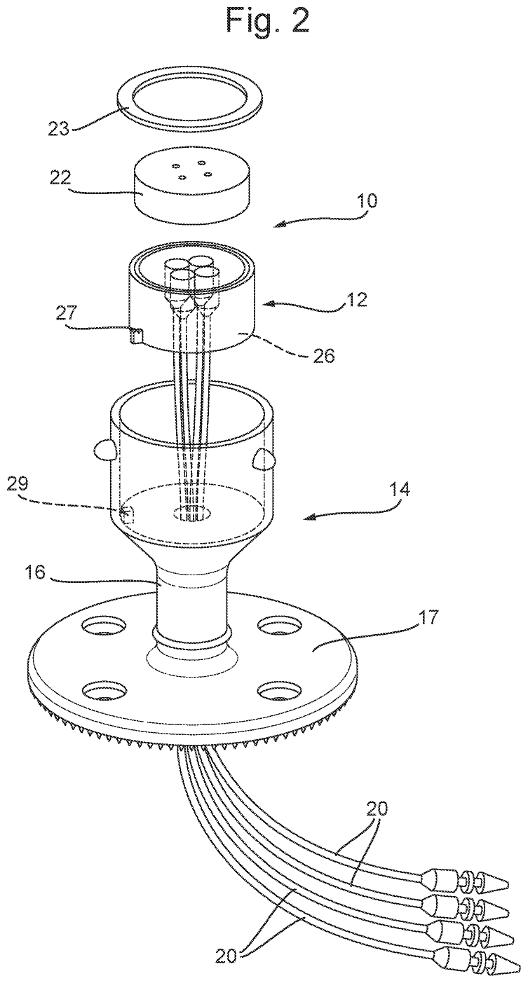

FIG. 2 is an exploded view of a device having multiple fluid ports;

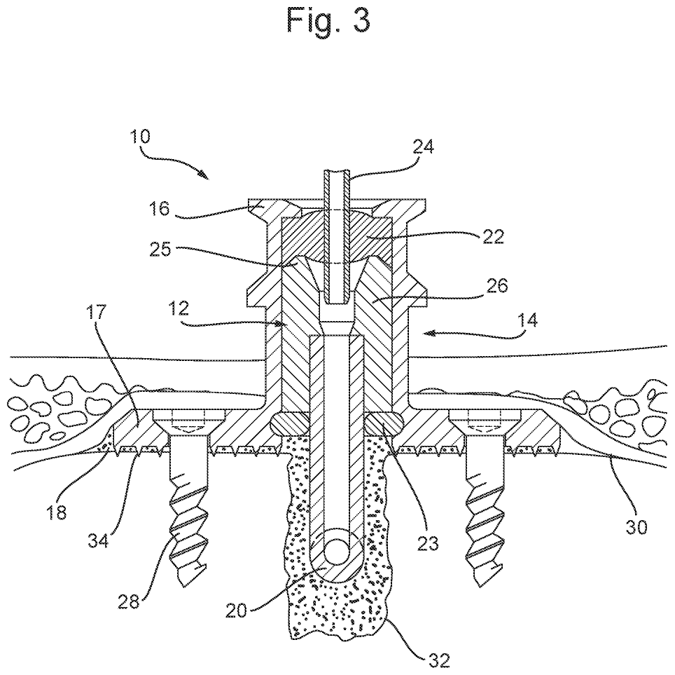

FIG. 3 is a cross sectional view of the device having a single fluid port of FIG. 1 when fixed to the skull;

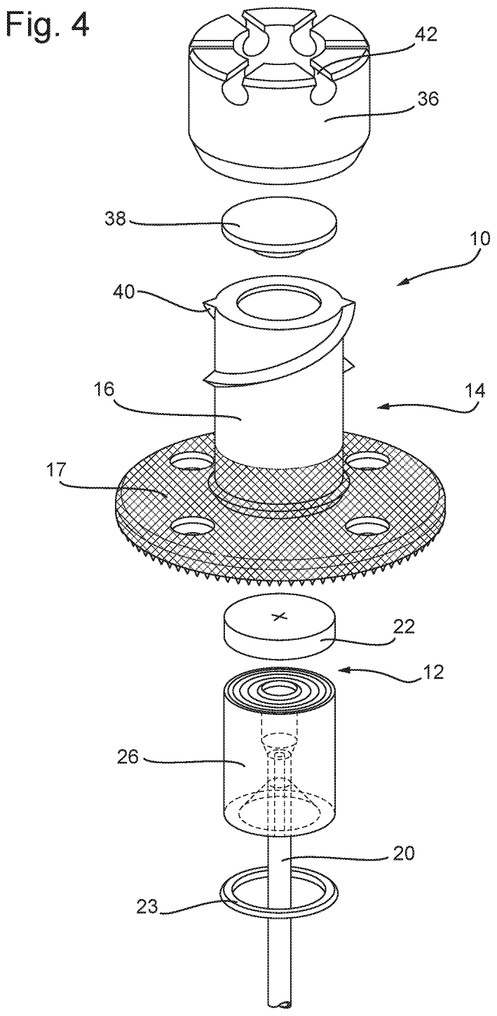

FIG. 4 is an exploded view of the device having a single fluid port of FIGS. 1 and 3;

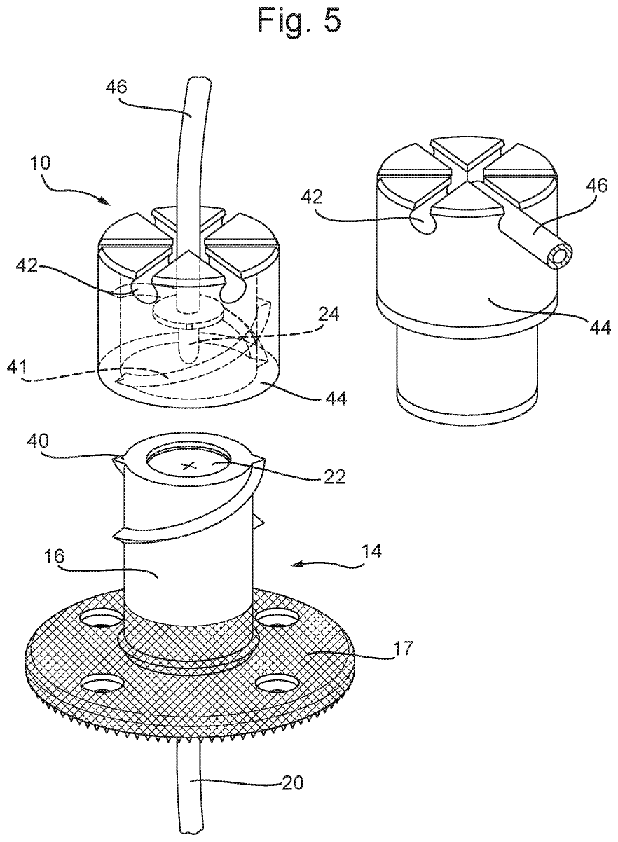

FIG. 5 is an exploded view showing a connector for a device having a single fluid port;

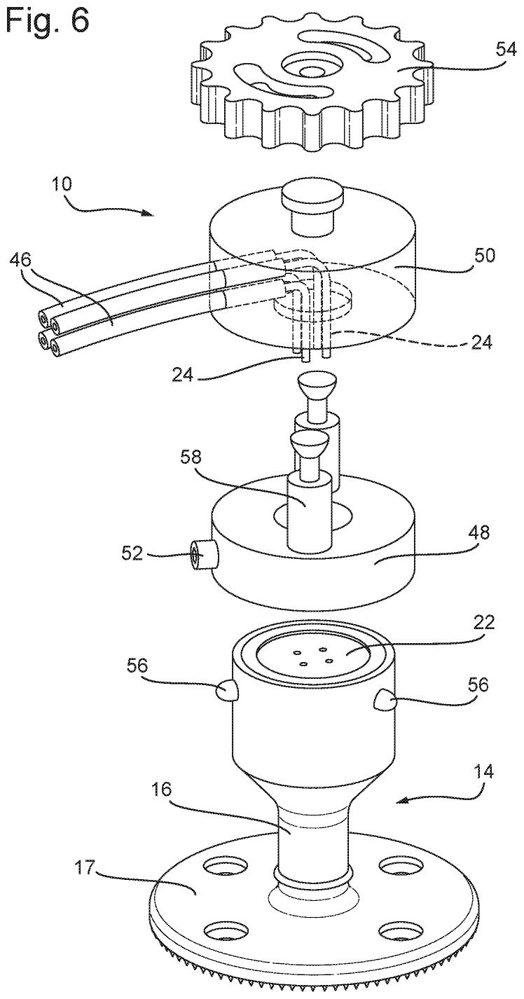

FIG. 6 is an exploded view showing a connector and guide member for the device having multiple fluid ports and fluid tubes shown in FIG. 2;

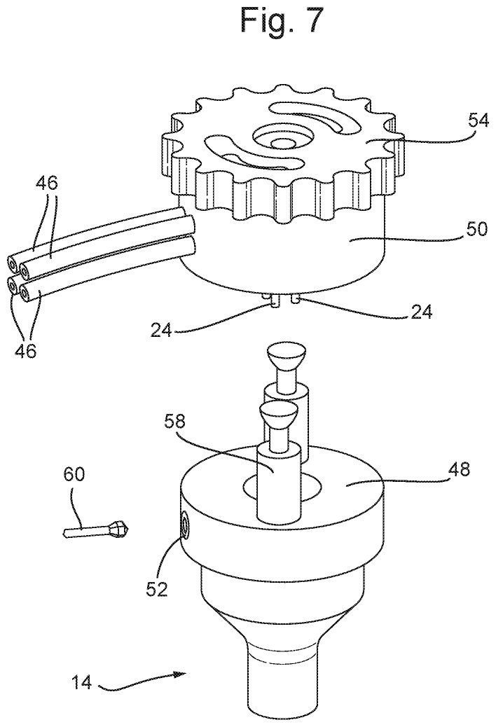

FIG. 7 is a perspective view showing attachment of the guide member to the housing of a device having multiple fluid ports;

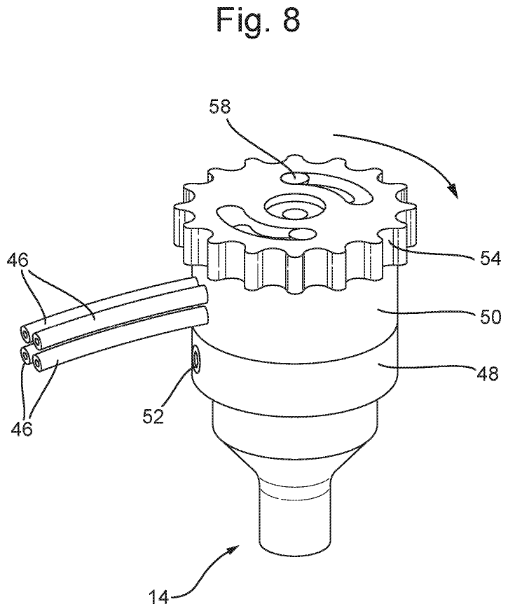

FIG. 8 is a perspective view showing attachment of the connector to the housing of the device having multiple fluid ports using the guide member;

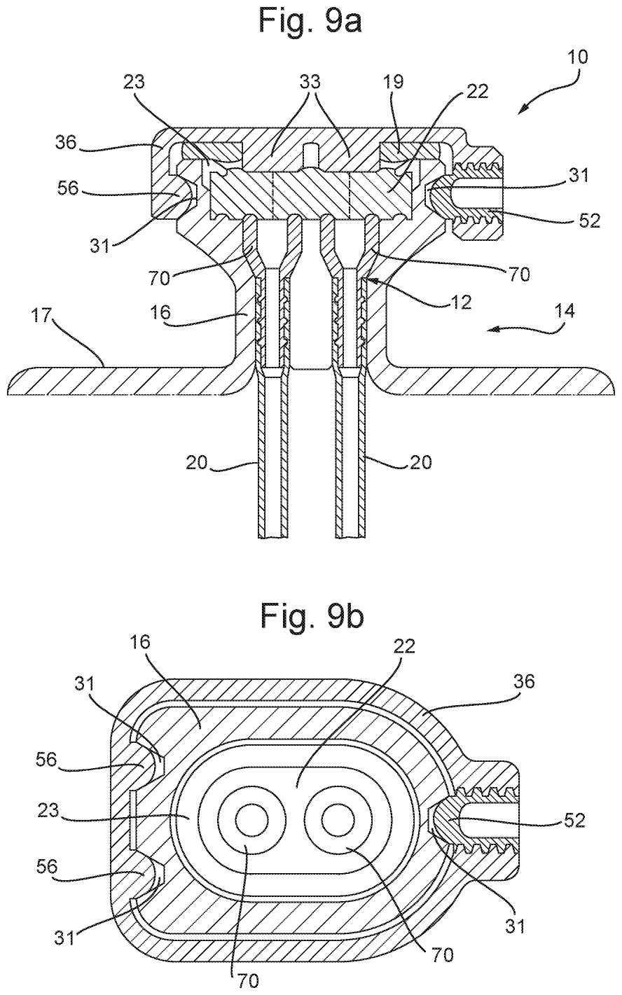

FIG. 9 shows cross-sectional and top-down views of a device having two fluid ports for use in intrathecal delivery when engaged with the cap;

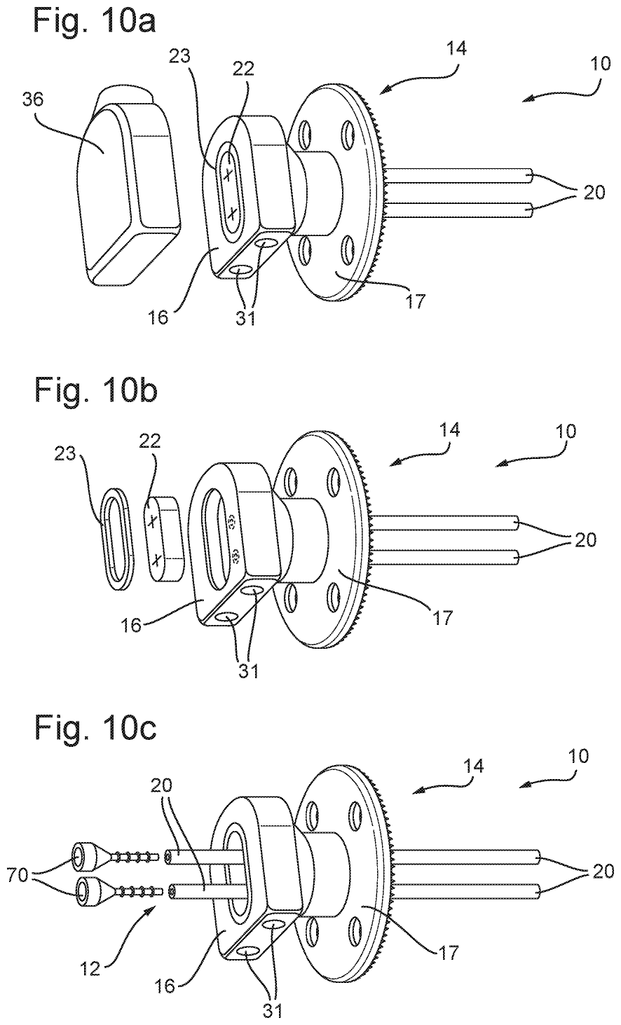

FIG. 10 shows various exploded views of the device having two fluid ports of FIG. 9;

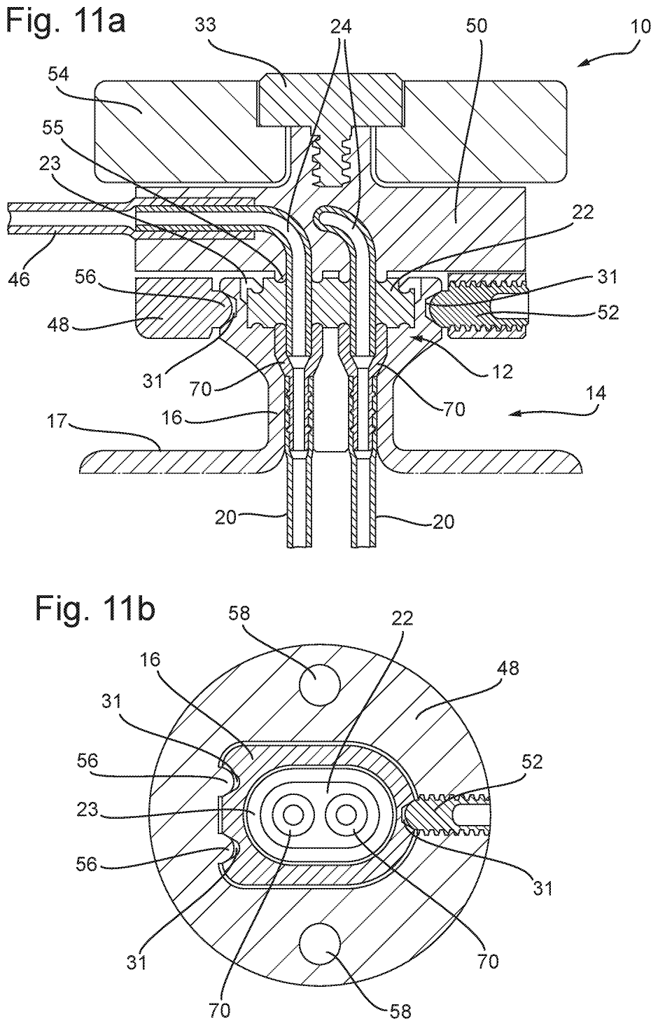

FIG. 11 shows cross-sectional and top-down views of the device of FIG. 9 when engaged with the guide member and connector;

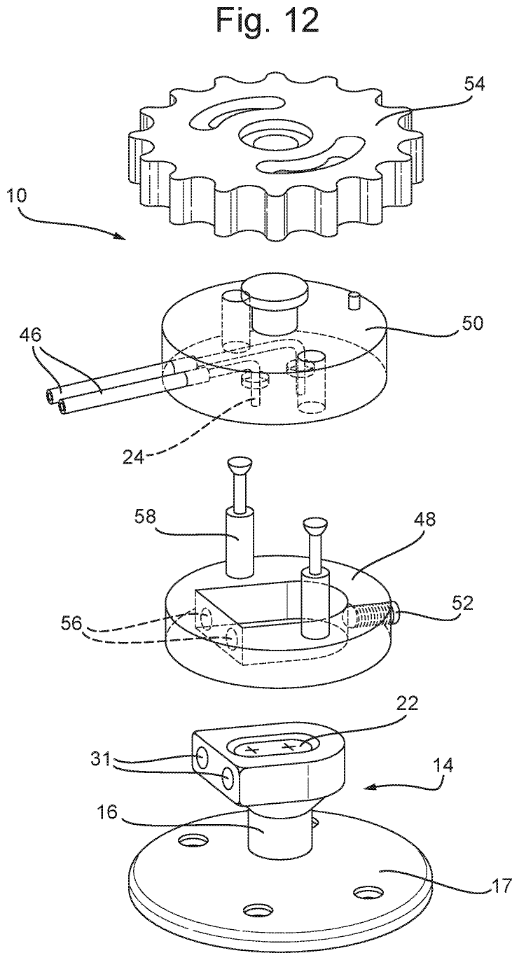

FIG. 12 is an exploded view showing a connector and guide member for the device having two fluid ports and fluid tubes shown in FIG. 9;

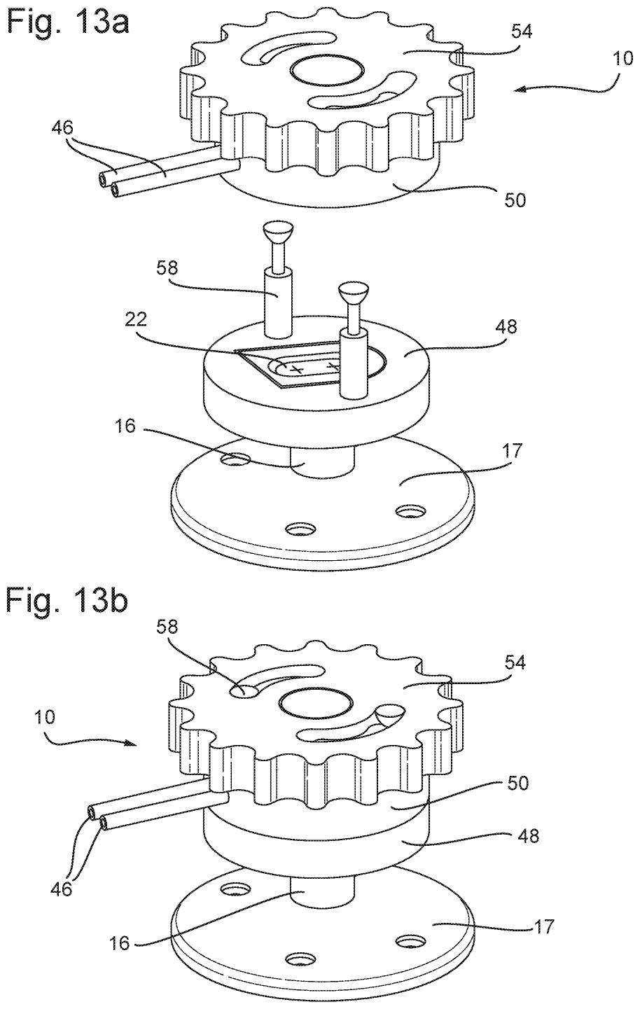

FIG. 13 is a perspective view showing attachment of the connector to the housing of a device having two fluid ports using the guide member;

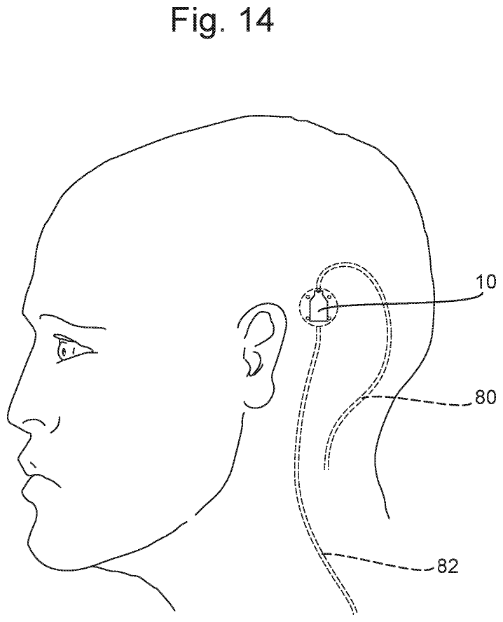

FIG. 14 illustrates the placement of the device when used for intrathecal delivery of therapeutic agents;

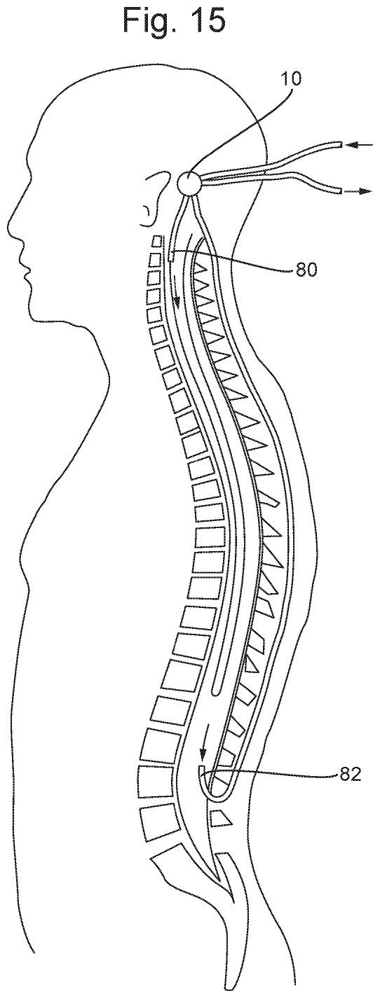

FIG. 15 illustrates the use of the device for intrathecal delivery of therapeutic agents;

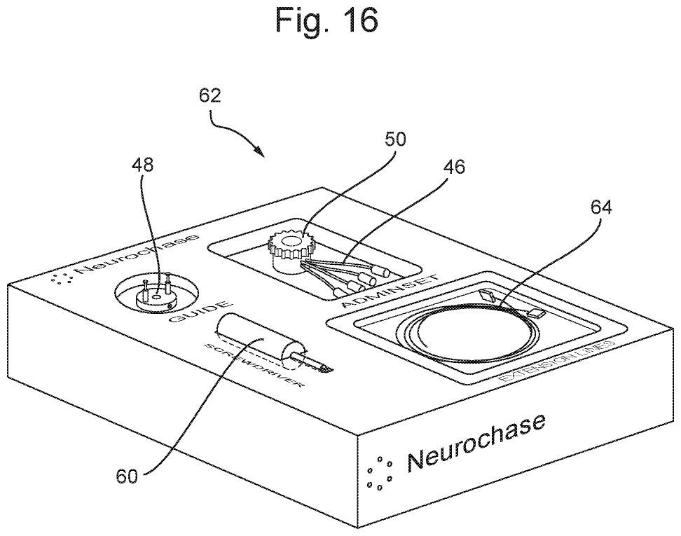

FIG. 16 shows a kit comprising the guide member and connector of FIGS. 6 to 8;

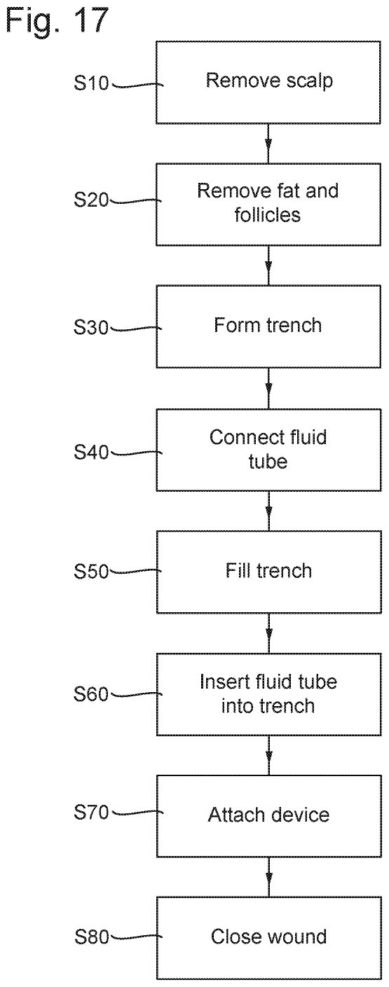

FIG. 17 is a flowchart of a method of implantation of a device for providing fluid access; and

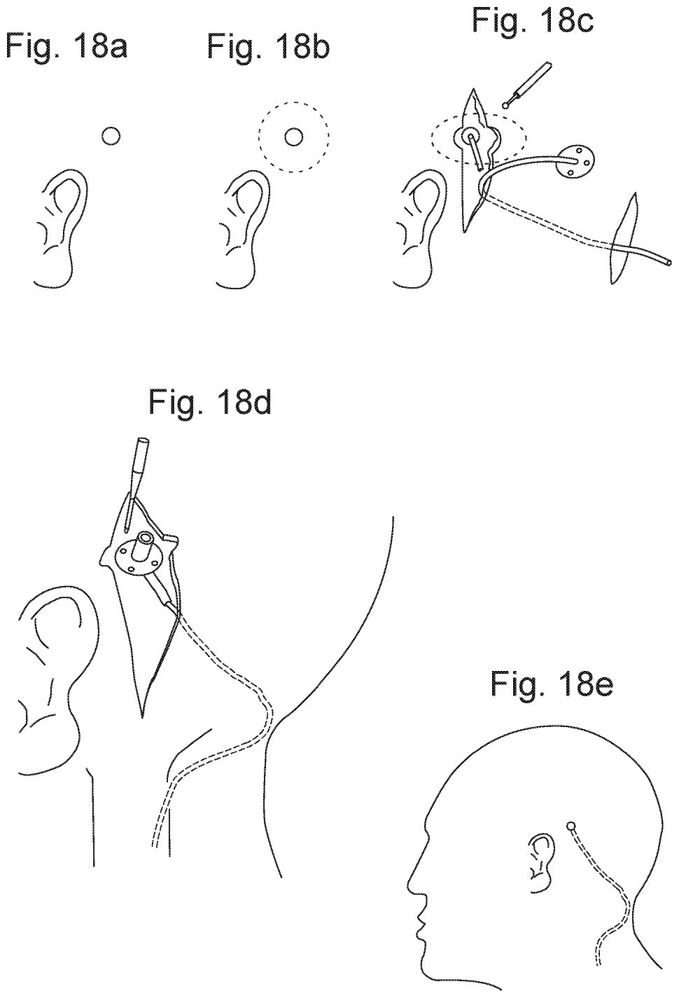

FIG. 18 illustrates some steps of the method of implantation of the device for providing fluid access.

FIG. 1 shows a device 10 for providing fluid access to the central nervous system (CNS) of a mammal. The device 10 provides a percutaneous, fluid-transferring device through which repeated access for the removal or delivery of fluid to the CNS can be gained. As discussed above, the fluid access provided by such a device 10 can be used for the treatment or diagnosis of neurological diseases. The device 10 is particularly suited for use in delivering therapeutic agents to the CNS, either directly into the brain parenchyma using the method of convection enhanced delivery (CED), or by infusion into the cerebrospinal fluid (CSF).

The device 10 comprises a fluid port 12 allowing the delivery or removal of fluid from the central nervous system, a housing 14 comprising an extracorporeal portion 16 allowing access to the fluid port 12, and a fluid tube 20 connected to the fluid port 12. The housing 14 may further comprise a lowermost surface 18 configured to engage with an outermost surface of the skull of the mammal. In the context of the device 10, the direction ‘lower’ or ‘below’ refers to a direction towards the interior of the skull when the device 10 is engaged with the outermost surface of the skull. This direction may also be referred to as the distal direction, i.e. such that the lowermost portion 18 is at the distal end of the housing 14.

Following implantation of the device, the fluid port 12 is connected to an implanted cannula or catheter via the fluid tube 20. The implanted cannula is typically placed within the CSF (either intraventricular or intrathecal), allowing infusion of fluid into the CNS using the device 10 via the fluid port 12 and fluid tube 20. The fluid port 12 can be connected to an extracorporeal infusion line, which may connect to a reservoir or any other suitable source or drain of fluid such as a syringe pump.

As shown in FIG. 2, the device 10 may comprise plural fluid tubes 20. Here, each fluid tube 20 is independently accessible via the fluid port 12. Following implantation, each fluid tube 20 would be connected to its own corresponding implanted cannula or catheter. This allows for fluid access to different regions of the CNS or for infusion of multiple different fluids. Alternatively, the fluid tube 20 may directly deliver fluid to the CNS, rather than being fluidly connected to the CNS via a catheter. The fluid port 12 may comprise one or more filters to filter fluid passing through the fluid port 12. For example, the filter may comprise bacterial filters and/or gas filters to prevent the introduction of bacteria or gas into the CNS.

The plural fluid tubes 20 may allow different therapeutic agents may be administered to different regions of the brain, or the same therapeutic agent to be administered to multiple regions. The embodiment shown in FIG. 2 is particularly suited to this type of application, having four fluid tubes 20 of smaller diameter.

The plural fluid tubes 20 may also be used for intrathecal delivery, for example by draining and/or circulating CSF. Circulation of CSF could be used to ensure a more uniform concentration of a therapeutic agent in the CSF when administering to the CSF, or to filter or replace CSF in the treatment of diseases such as meningitis. For example, as shown in FIG. 15, one fluid tube 20 may be connected to a first catheter 80 inserted into the cisterna magna or ventricle of the brain for infusion of the therapeutic agent, and a second fluid tube 20 connected to a lumbar catheter 82 inserted into the spinal column for drainage of CSF. This can allow for mixing of a therapeutic agent with the CSF, and closed cycling of the CSF-agent mixture to ensure a uniform distribution of the therapeutic agent through the CNS, and/or filter cellular debris from the CSF. Other applications include infusion of therapeutic agent both above and below an obstruction in the CSF, and/or delivery of multiple drugs simultaneously. Such an arrangement can also allow chronic and/or intermittent infusion into the CSF and sampling of CSF, as well as ambulatory infusions. This reduces discomfort and the chance of complications such as infection compared to existing methods such as lumbar puncture or intraventricular injection.

The advantages of a transcutaneous septum-sealed device as described herein that provides fluid access to the CNS include the facility to deliver therapies or inert fluid directly to the brain or spinal cord parenchyma and/or to the cerebrospinal fluid continuously or intermittently over hours, days, weeks, months, or years without the need for repeated surgical procedures. The device also facilitates the intermittent removal of CSF or fluid from the CNS parenchyma including fluid from tumour, developmental, or infected cysts, also without the need for repeated invasive procedures.

CNS disorders that may be treated with therapeutic agents delivered through the device include (but are not limited to) neurodegenerative disease, movement disorders, an enzyme deficient condition, a neuroinflammatory disease, CNS infection, an acquired neurological injury, epilepsy, cancer, sub arachnoid haemorrhage and cerebral vasospasm.

Neurodegenerative diseases include dementia, Lewy body disease, Alzheimer's disease, Huntington's disease, Amyotrophic Lateral Sclerosis (ALS), Multiple System Atrophy, Spinal muscular atrophy, Friedreich's Ataxia, Huntington's disease, Parkinson's disease, Parkinson's plus syndromes, and Corticobasal degeneration. Enzyme deficient conditions include Lysosomal Storage diseases, Tay Sachs Disease, Sandhoff Disease, Neuronal Ceroid Lipofuscinosis, Niemann Pick disease type-C, Hunter Syndrome, Hurler disease and Gaucher's Disease. Neuroinflammatory diseases include Multiple Sclerosis and prion diseases. CNS infections include meningitis cerebritis and cerebral abscess. Acquired neurological injuries include stroke, traumatic brain injury or spinal cord injury. Cancer may include leptomeningeal carcinomatosis or brain cancer. Brain cancer may be characterised by the presence of primary or secondary brain tumours. The primary brain tumour may be an astrocytoma, such as glioblastoma multiforme (GBM), and may be diffuse intrinsic pontine glioma (DIPG).

By way of example the therapeutic agent may include (but is not limited to) neurotrophins, histone deacetylase inhibitors, gene therapies, enzymes, immune-therapy, SiRNAs, antisense oligonucleotides, chemotherapy, Auger electron emitters, immunotoxins, molecular targeted therapies, monoclonal antibodies, oncolytic viruses, viral vectors, chemotherapy agents, nanoparticles, such as gold or iron nanoparticles, antispasmodics, thrombolytics and botulinum toxin.

The therapeutic agent may be administered in the form of a pharmaceutical composition, which may comprise any pharmaceutically acceptable carrier, adjuvant or vehicle. Pharmaceutically acceptable carriers, adjuvants and vehicles that may be used in the pharmaceutical compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and polyethylene glycol.

The therapeutic agent may be administered in an inert diluent such as artificial CSF. An inert fluid such as artificial CSF, may be infused directly into the subarachnoid space or ventricle to replace endogenously produced CSF or infused into the brain parenchyma prior to and/or following the infusion of a therapeutic agent or be co-infused with a therapeutic agent that is delivered through a separate cannula.

Use of the device to intermittently infuse therapies directly into target volumes in the brain parenchyma through chronically-implanted fine cannulas has advantages in the treatment of neurological diseases. The blood brain barrier prevents many therapies from reaching their therapeutic targets in the CNS when delivered into the systemic circulation and for the treatment of many CNS diseases therapy needs to be confined to specific brain volumes to limit potential side effects. The infusion of therapies directly into to the brain parenchyma using the method of convection enhance delivery (CED) can achieve homogenous and precise dosing of selected treatment volumes and achieve long tissue exposure times because the blood brain barrier now acts to retain the therapy in the brain. The long biological half-life in the CNS when delivered by CED means that repeated infusions to maintain a therapeutic effect can be days, weeks or months apart. The device facilitates intermittent infusions without the need for repeated and potentially risky surgery. The device also enables dosing to be modulated according to patient response, or side effects, which may not otherwise be feasible with a one-off gene therapy for example. Examples of the device's potential use for intermittent infusions to the brain parenchyma include:
1. Intermittent infusions of neurotrophins such as Glial cell-line Derived Neurotrophic Factor (GDNF), or Cerebral Dopamine Neurotrophic Factor (CDNF) to the putamen via two or four cannulas to achieve neuro-restoration in Parkinson's disease. Phase II trials of treating 42 patients with Parkinson's disease on a monthly basis for over 18 months with infusions of GDNF to the putamen and 17 patients with CDNF with a similar regimen via a transcutaneous port have demonstrated that this mode of treatment is safe and effective (Whone A. et al, Brain, Volume 142, Issue 3, 1 Mar. 2019).
2. Intermittent infusions of antisense oligonucleotides (ASOs) targeting the huntingtin protein RNA (HTT RNA) to treat Huntington's disease. This may be achieved via cannulas placed in the putamen and the caudate nuclei. This method of treating Huntington's disease may be more effective in achieving therapeutic concentrations of the ASO's in the caudate and putamen, the most affected structures in the disease, than by delivery into the CSF which is the method that has been previously employed.
3. Histone deacetylase inhibitors (HDACis) have been shown to be neuroprotective in several neurodegenerative disease models including models of Parkinson's disease, Huntington's disease, Alzheimer's disease, Spinal Muscular atrophy and amyotrophic lateral sclerosis (ALS). HDACis have ubiquitous effects and the majority do not cross the blood brain barrier. Intermittent infusions of HDACis to relevant CNS targets may be achieved using the device.
4. The treatment of epilepsy, movement disorders, with the direct infusion of Botulinum toxin into targeted volumes of the brain parenchyma has been described (US20080160121). Botulinum toxin when infused into the brain parenchyma reversibly inhibits the release of neurotransmitters including acetylcholine, norepinephrine, and glutamate for a period of 2 to 6 months controlling symptoms for this period. Intermittent infusions of Botulinum toxin into the epileptogenic tissue via the device, for example in the medial temporal lobe of patients with medication resistant temporal lobe epilepsy, may be an effective long-term treatment. Use of the device to intermittently infuse Botulinum toxin into functional targets in the brain to make reversible lesions may be effective in controlling movement disorders such as dystonia, dyskinesia and Parkinson's; and infusions into the globus pallidus, the Ventral Intermediate nucleus of the thalamus (VIM) for the control of tremor. Reversible functional lesions with intermittent infusions of Botulinum toxin into the anterior cingulate gyrus using the device could also be used to control chronic pain. Likewise reversible functional lesions with intermittent infusions of Botulinum toxin delivered through the device could be used for the treatment of major psychiatric disorders including depression targeting the subcallosal cortex, or the ventral capsule/ventral striatum, and for obsessive compulsive disorders by targeting the ventral capsule/ventral striatum, for example.
5. Repeated administration of drugs by CED to the same target volume without the need for further surgery. This is especially important when treating malignant brain tumours, because repeated exposure to chemotherapy is essential to ensure that cells are adequately exposed to the drug. Monthly infusions of a combination of a HDACi, Sodium Valproate and Carboplatin to the pons in children with Diffuse Intrinsic Pontine Gliomas through a septum sealed transcutaneous port has been shown to both safe and effective (Szychot E. et al, Int. J of Clinical Oncology, 2021). Similarly monthly infusions of Carboplatin to the brain parenchyma via a transcutaneous septum sealed port have been shown to effectively control recurrent glioblastoma (Barua N U, et al. Drug Delivery 2016).

Examples of the use of the device to deliver treatments to the CNS by providing fluid access to the cerebrospinal fluid (CSF) include:
1. The treatment of leptomeningeal carcinomatosis. This is a complication of cancer in which the disease spreads from the original tumour site to the meninges surrounding the brain and spinal cord. 18.1 million people are diagnosed with cancer every year, and although cancer survival rates are improving, there are increasing numbers with leptomeningeal carcinomatosis. Leptomeningeal carcinomatosis occurs in 5-8% of solid tumours (including: 40% of patients with breast cancer, 20% with lung cancer and 10% with melanoma); 5-15% of haematological tumours and 10-32% of primary CNS tumours. It is usually fatal within 3 to 6 months. Current treatment options for Leptomeningeal carcinomatosis include the delivery of chemotherapy via a lumbar puncture or intraventricular injection via an Ommaya reservoir. For example, for a patient with leukaemia, this may include methotrexate 15 mg daily for 5 days every 2 weeks or cytarabine 30 mg daily for 3 days. However, numerous problems are associated with delivering intrathecal therapy by lumbar puncture. It is painful (general anaesthetic being required for children) and difficult to perform. The process is traumatic and carries a risk of infection. No effective circulation of CSF in the spinal theca results in heterogenous drug concentration, which can lead to local drug toxicity due to high local concentration at the injection site, and underdosing at regions distant from the injection site. Complications include seizures, arachnoiditis, motor and sensory deficits, headaches, nausea, vomiting, necrotising leukoencephalopathy. CSF obstruction above the infusion site can lead to untreated regions, and the volume of infusate is limited. Intraventricular injection into the Ommaya reservoir also has similar problems, along with a high rate (approximately 10%) of infection and/or catheter misplacement. Use of the trans-cutaneous septum sealed device to deliver chemotherapy into the CSF to treat leptomeningeal carcinomatosis facilitates repeated access to the CSF which is painless and sterile and will also facilitate continuous ambulatory infusions through a portable infusion pump. This will help to maintain therapeutic concentrations of the therapy in the CSF for prolonged periods. If a dual catheter device is employed with one catheter delivering the chemotherapy to the region of the cisterna magna or ventricle, for example, then through a second catheter in the lumbar theca, CSF can be intermittently withdrawn to determine the concentration of the therapy in the CSF. A device with dual catheters, one implanted in the cisterna magna or ventricle and a second in the lumbar theca can be used to deliver therapy in combination with artificial CSF through the former and drain CSF through the latter. This will ensure that the therapeutic dose can be homogenously distributed through the CSF space and tumour debris in CSF can be removed. A dual catheter system as described can also be used to deliver chemotherapy above and below a tumour obstructing the CSF pathways.
2. Providing fluid access to the CSF through two or more catheters wherein artificial CSF can be infused through one catheter and CSF withdrawn through a second catheter to wash out blood or pathogens from the CSF. Such an approach may be beneficial in the treatment of sub arachnoid haemorrhage to remove blood products from the CSF that can cause cerebrovascular spasm and stroke. This may include the co-infusion of thrombolytic agents to accelerate the clearance of blood clots. The device may also be used to deliver antispasmodic drugs to the CSF such as Nimodipine to counteract cerebral vasospasm. In some cases of meningitis, the device could be used to deliver artificial CSF through one catheter and drain infected CSF through a second. It would also provide the means to deliver antibiotics or antiviral agents to the CSF and maintain tightly controlled concentrations which could be monitored with regular sampling through a second catheter in the CSF space.
3. Infusion of antisense oligonucleotides into the CSF. Such infusions have shown efficacy in experimental models of Alzheimer's disease, Frontotemporal dementia, Huntington's disease, Amyotrophic Lateral Sclerosis (ALS) and Spinal Muscular Atrophy (SMA). Treatment for SMA with intermittent infusions into the CSF via lumbar punctures has been approved for clinical use. The transcutaneous septum sealed device described herein would be an alternative means of delivery with the benefits of this approach as described herein.

The device 10 of FIG. 1 or FIG. 2 may comprise a seal sealing the fluid port 12. The seal may comprise a valve. The valve may be activated or opened when connected to a corresponding reciprocal connecting member. For example, the valve may be a mechanical valve, which requires physical activation of the valve to permit fluid flow. The valve may be self-sealing so that it closes automatically upon removal of the connecting member. Alternatively, the valve may be a pressure-sensitive valve, which is operable to open upon fluid flow and to close when fluid flow stops. The pressure-sensitive valve may comprise a split seal or split septum.

In FIG. 1 and FIG. 2, the seal is a septum 22 sealing the fluid port 12. The septum 22 may be accessed with a hollow needle 24 or a blunt hollow cannula. In the latter case, the septum 22 is preferably a pre-pierced or split septum. The septum 22 may comprise medical grade silicone. Where the device 10 comprises plural fluid tubes 20, the septum 22 may seal a single one of the fluid tubes 20, or may seal plural ones of the fluid tubes 20. The septum 22 may have a diameter of 1-10 mm, preferably 2-5 mm, for example 3 mm. The septum may have a thickness of 0.5-5 mm, preferably 1-3 mm, for example 1.5 mm. However, the septum 22 is not limited thereto, and any suitable dimensions may be used depending on the configuration of the device 10. The housing 14 may further comprise a retaining member 23, such as a press-fit ring, to retain the septum in the desired position within the housing. The retaining member 23 may be made of plastic (for example polyetheretherketone) or metal, for example titanium.

The housing 14 preferably comprises plastic (such as polyetheretherketone) or metal, for example titanium, both of which are strong, lightweight, and biocompatible. The housing 14 may be formed by any suitable production method such as moulding, casting, or milling. Preferably, the housing 14 is formed by 3D printing. The housing 14 may comprise a moulded portion 26 within the housing 14 and into which other components of the device 10 (such as the fluid tube 20) are fitted. The moulded portion 26 may comprise plastic, for example polyetheretherketone (PEEK) or carbothane. The moulded portion 26 may comprise an alignment feature such as a recess 27 (see FIG. 2), which cooperates with a corresponding alignment feature 29 inside the housing 14 to ensure that the moulded portion is correctly oriented when the device 10 is assembled.

The housing 14 may comprise one or more protrusions 25 configured to compress the septum 22. Optionally, the protrusions 25 are provided by the moulded portion 26. The protrusions 25 may be configured to produce focal compression on the septum 22. The protrusions 25 may be configured to produce compression localised at the entrance to the fluid port 12. The protrusions 25 may compress the septum 22 around a region of the septum 22 where the needle 24 is advanced through the septum 22. For example, where the septum 22 is a pre-pierced or split septum, the region may include the piercing or split in the septum 22. The protrusions 25 may compress the septum 22 from a side of the septum 22 that faces the interior of the housing 14. The protrusions 25 may take the form of an annular ridge around the region where the needle 24 will be advanced through the septum 22.

Fluid from the needle 24 may be supplied at higher pressure than the typical intracranial pressure during infusions, in order to force the fluid into the CNS. Consequently, the septum 22 may not ordinarily provide sufficient sealing force around the needle to prevent leakage of fluid. The protrusion 25 act as a valve seating that compresses the septum 22 around where the needle 24 penetrates the septum. The compression of the septum 22 ensures a liquid-tight seal to prevent leakage of fluid during administration or removal of fluid using the device 10. Where the device 10 includes plural fluid tubes 20 intended for access using separate needles 24, protrusions 25 may be provided around each of the regions of the septum 22 through which a needle 24 will be advanced. In this case, the compression of the septum 22 also prevents cross-leakage or contamination between the plural fluid tubes 20.

The needle 24 may comprise a hub 21 configured to engage with the proximal (exterior) face of the septum 22 and compress the septum 22 when the needle 24 has been advanced to its predetermined position. When the needle 24 comprises a hub 21, the protrusions 25 preferably have a corresponding shape and size to the hub 21, such that the area of the septum 22 compressed by the protrusions 25 is similar to, and opposed to the hub of the respective needle 24. This will assist in creating a tight seal around the needle 24 during fluid transfer.

The extracorporeal portion 16 allows access to the fluid port 12. Typically, the fluid port 12 is accessed from a proximal end of the extracorporeal portion 16. The extracorporeal portion 16 may have a cylindrical shape and pass through an opening in the skin. The extracorporeal portion 16 may have a diameter of 1-10 mm, preferably 2-6 mm, more preferably approximately 4 mm. The extracorporeal portion 16 may have a height of approximately 1-15 mm, preferably 2-10 mm, more preferably 6 mm. However, the extracorporeal portion 16 is not limited thereto, and any suitable dimensions may be used depending on the configuration of the device 10.

In addition to the extracorporeal portion 16, the device 10 may comprise a subcutaneous portion 17 having a larger diameter than the extracorporeal portion 16 and provided at a distal end of the housing 14. In FIG. 1, the subcutaneous portion 17 is disc-shaped, and typically has a thickness of 0.5-3 mm, preferably approximately 1 mm, but in general it may have any suitable shape. The lowermost surface 18 may be provided by the subcutaneous portion 17.

A surface of the housing 14 configured to be in contact with tissue of the mammal may have at least one of a texture and coating configured to promote tissue integration. Tissue integration in this context may include osseointegration of the lowermost surface 18 with the skull, as well as integration of soft tissue with other surfaces of the housing 14. It may be particularly advantageous to provide the texture and/or coating on surfaces of the housing 14 other than surfaces of the extracorporeal portion 16, such as surfaces that are configured to be located under the skin and/or in contact with soft tissue following implantation. This preferably includes surfaces of the subcutaneous portion 17. Suitable textures and coatings include a microporous surface, 3D-printed textured surface, a coating of plasma sprayed titanium, and/or a hydroxyapatite coating. Tissue integration promotes a sealed tissue-device interface, which can prevent ingress of bacteria. This reduces the likelihood of persistent or recurring inflammation and/or infection of the soft tissues around the device 10, which can cause significant morbidity.

The one or more fluid tubes 20 are connected to the fluid port 12 and preferably extend below the housing 14 through the lowermost surface 18. The fluid tube 20 is preferably made from a flexible material to allow it to be positioned easily during implantation. An example of a suitable material for the fluid tube 20 is a low protein-binding polyurethane such as carbothane, or alternatively PEEK. The fluid tube 20 may have an outer diameter of 0.2-3 mm, preferably approximately 1 mm or approximately 1.5 mm. The fluid tube 20 may have an inner diameter of 0.1-1 mm, preferably approximately 0.2 mm or approximately 0.7 mm. Different dimensions may be preferred for different applications.

In some situations, the device 10 may be used for administering therapeutic agents to one or more regions of the brain, for example by CED. In this situation, it is preferable that the fluid tubes 20 have a smaller diameter. The smaller diameter allows more fluid tubes 20 to be included in the same device 10 and to fit into the trench 32. It also minimises the dead volume of the fluid tubes 20, which can reduce wastage of the therapeutic agent during each administration due to unused agent left in the fluid tube 20. For such applications, the fluid tubes 20 may have an outer diameter of 0.5-2 mm, preferably approximately 1 mm. For such applications, the fluid tubes 20 may have an inner diameter of 0.1-0.5 mm, preferably approximately 0.2 mm.

In some situations, the device 10 may be used for draining and/or circulating CSF. In this situation, it is preferable that the fluid tubes 20 have a larger diameter to allow the CSF to be circulated and/or drained at a suitable rate. For such applications, the fluid tubes 20 may have an outer diameter of 0.5-3 mm, preferably 0.6 to 2.5 mm, more preferably approximately 1 mm. For such applications, the fluid tubes 20 may have an inner diameter of 0.3-1 mm, preferably 0.5-0.7 mm. For example, the inner diameter may be at least 0.4 mm, optionally at least 0.5 mm, further optionally at least 0.6 mm. The inner diameter of the fluid tube 20 may be of sufficient size to permit free flow of CSF of at least 10 ml per hour.

FIG. 3 shows the device 10 in an implanted state. The lowermost surface 18 is configured to engage with an outermost surface 30 of the skull. The device 10 may thus be fixed or mounted to the outermost surface 30 of the skull. In FIG. 3, the device 10 is attached by the lowermost surface 18 being configured to engage with, and attach to, the outermost surface 30 of the skull using a plurality of screws 28. The screws 28 secure the device 10 to the outermost surface 30 via two or more holes in the subcutaneous portion 17. The screws 28 in FIG. 3 have a diameter of 2 mm, but any suitable diameter of screw may be used depending on the requirements of the device 10, for example screws 28 having a diameter of 1-3 mm. Optionally, the lowermost surface 18 may be configured to engage with, and attach to, the outermost surface 30 of the skull by other means, for example using an adhesive.

Optionally, and as shown in FIGS. 1-3, no part of the housing 14 extends below the lowermost surface 18. Thus, only the tips of the screws and fluid tube 20 extends below the lowermost surface 18 of the housing 14, and into the outermost surface 30 of the skull. This configuration provides the advantage over prior art devices that there is no need for accurate machining of the skull to create a precisely profiled hole to accommodate the device. This would typically be carried out using stereotactic or robotic guidance and the use of a skull mounted jig with a series of specialist cutting tools. Mounting the device on the skull surface significantly simplifies the method of implantation, and reduces surgical procedure time. This in turn reduces costs and the associated risks to the patient of prolonged anaesthesia and infection. Further, the device 10 can be installed in regions of the skull where the bone is thin, which may not be possible with prior art devices that require a recess of a particular depth to be machined in the skull. Similarly, the device can be installed in the skull of a child or baby, which may have very thin skulls.

As shown in FIG. 3, the fluid tube 20 may be configured to bend so as to run along a trench 32 formed in the outermost surface 30 of the skull. Preferably the fluid tube 20 is bent by approximately 90°. The trench 32 may extend from an implantation site at which the device 10 is implanted, extending from beneath the lowermost surface 18. The fluid tube 20 extends below the housing 14 through the lowermost surface 18 and into the trench 32. By bending and running in the trench 32, the fluid tube 20 can exit from beneath the device 10 and connect to the corresponding implanted cannula or catheter. The fluid tube 20 is preferably configured to bend at a point below the lowermost surface 18, for example such that the fluid tube 20 enters the trench 32 at a point below the lowermost surface 18. This can further protect the fluid tube 20 and its entry point into the trench 32 so as to further reduce the risk of infection. The trench 32 is preferably filled with bone cement (e.g. an acrylic cement) before the fluid tube 20 is placed within it. The cement seals and retains the portion of the fluid tube 20 proximal to the device 10 in the desired position within the trench 32. Filling the trench 32 with bone cement also restores the integrity of the outermost surface 30 of the skull and reduces the risk of infection.

Accommodating the bend of the fluid tube 20 in the trench 32 formed in the outermost surface 30 of the skull, rather than accommodating it within the device 10 itself, has the advantage of greatly reducing the size of the device 10 both above and below the skull surface. This is advantageous when the skull is thin. For example, a child's skull has a typical thickness of 2 mm, and bulky devices which extend below the skull surface such as disclosed in the prior art will risk compressing the brain.

It is possible that an interior surface (the inner table) of the skull is breached when forming the trench 32. In this case, the fluid tube 20 may be partially below the interior surface of the skull, even where no part of the housing 14 is below the lowermost surface 18 of the device 10. However, it is preferable that no part of the device 10 extends through an interior surface of the skull when the lowermost surface 18 of the housing 14 is engaged with the outermost surface 30 of the skull. This reduces the chance of inflammation or infection to the brain.

In addition, configuring the fluid tube 20 to bend and run along the trench 32 means that the fluid tube 20 is immobilised in the trench for some distance under the skin away from the housing 14. This reduces movement of the fluid tube 20, which could disturb the skin in the immediate area around the device 10 and prevent sealing of the skin around the device 10. Thereby, the risk of infection and marsupialisation around the device 10 is greatly reduced. This is particularly true in comparison to some known devices in which a tube extends subcutaneously directly from the housing above the surface of the skull. To reinforce this advantage, optionally no part of the fluid tube 20 extends outside of the housing 14 (i.e. exits the housing 14 by passing through an exterior surface of the housing 14) except through the lowermost surface 18. Consequently, the device 10 is configured such that no part of the fluid tube 20 extends outside of the housing 14 (i.e. exits the housing 14 by passing through an exterior surface of the housing 14) above the outermost 30 surface of the skull when the device 10 is engaged with the outermost surface 30 of the skull.

As shown in FIG. 1 and FIG. 3, the lowermost surface 18 of the housing 14 may comprise a plurality of teeth 34 for engagement with the outermost surface 30 of the skull. The teeth 34 grip the outermost surface 30 of the skull and provide increased stability of the device 10. The inventors have recognised that integration of the skin's dermis into the device 10 is greatly enhanced when the device 10 and the skin are immobilised relative to one another. Integration of the dermis and relative immobilisation reduces the risk of marsupialisation around the device 10, which in turn greatly reduce the risk of complications such as infection. In addition, the teeth 34 accommodate unevenness of the outermost surface 30, and greatly reduce shearing action on the screws 28, or whichever attachment means is used to engage the lowermost surface 18 of the housing 14 with the outermost surface 30 of the skull. When combined with the feature that no part of the housing 14 extends below the lowermost surface 18 and fixation of the device 10 to the skull surface, for example with screws 28 through the subcutaneous portion 17, stability of the device 10 can be provided even in a thin skull.

The teeth 34 constitute a roughening of the lowermost surface 18 of the housing 14. Two or more teeth 34 may be provided. Preferably, the plurality of teeth 34 is distributed over the lowermost surface 18, for example being distributed over at least 50%, preferably at least 70%, more preferably at least 80% of an area of the lowermost surface 18. The plurality of teeth may comprise a large number of small teeth 34. The teeth 34 may be sharpened to facilitate engagement with the outermost surface 30 of the skull. The teeth 34 may have a height (i.e. a distance of extension by which they extend from the lowermost surface 18) of less than 3 mm, preferably less than 1 mm. The teeth 34 are driven into and penetrate the outermost surface 30 of the skull when the bone fixation screws 28 are tightened, thereby locking the device 10 into the outermost surface 30 at the desired location. As well as improving the immediate stability of the device 10, penetration of the outermost surface 30 by the teeth 34 stimulates bone integration for long-term stability.

The device can engage the skull via a layer of acrylic cement. The acrylic cement can act to fill gaps between the teeth 34, and generally between the lowermost surface 18 and the skull outermost surface 30, and provide a further adhesive effect. The acrylic cement can also provide a sealing effect when used in this manner.

FIG. 4 shows an exploded view of an embodiment of the device 10 in which the device 10 comprises a septum 22 sealing the fluid port 12, and in which the device 10 further comprises a cap 36. The cap 36 is configured to engage with the extracorporeal portion 16 of the housing 14 and compress the septum 22 when the cap 36 is attached to the device.

Septum-sealed devices in general may be configured to apply a high compressive pressure to the septum to ensure that the septum seals adequately once the needle is removed. This high compressive pressure is applied by the device housing radially in the plane of the septum (i.e. perpendicular to the direction in which a needle is intended to be inserted through the septum). This means that the septum will be tightly pressed against the diameter of any needle pushed through the septum, thereby ensuring sealing around the needle. Such high compressive pressure in the septum may however impede its radial displacement as a hollow needle is passed through it, resulting in the hollow needle coring septum material that blocks the needle or port. Thus, high compressive forces (which themselves are useful for ensuring the sealing effect of the septum) can lead to damage to the septum when it interacts with a needle. In addition, the compressive force on the septum material at the entrance to a port sealed by the septum is substantially increased when a hollow needle is passed through the septum and into the port. This can result in septum material being forced into the port. Fragments of septum material may be sheared off and potentially obstruct fluid flow in the port. Shearing forces on compressed septum material from the repeated passage of needles through it will produce wear debris that will over time impede flow in the port or be transported into the CNS potentially causing local inflammation. Degradation of a compressed septum by the repeated passage of needles will also reduce its effectiveness as a fluid and hermetic seal, posing a risk to patients and shortening the life of the device.

To address these problems with septum-sealed devices, the present device 10 may include the cap 36 configured to engage with the extracorporeal portion 16 of the housing 14 and compress the septum 22 when the cap 36 is attached. The cap 36 is configured to compress the septum 22 by applying a force perpendicular to a plane of the septum 22, i.e. parallel to a direction in which a needle is intended to be inserted through the septum 22. Thereby, the device 10 provides a compression force on the septum 22 using the protective cap 36, rather than exclusively by compressing the septum 22 as it is installed in the device 10 (for example using solely radial compression). This means that the septum 22 can be thinner and more compliant whilst maintaining an effective seal (preventing fluid leakage or air ingress) when the cap 36 is applied. The septum 22 may be under tension and/or compression only from the cap 36, i.e. no significant compression and/or tension force is applied to the septum 22 when the cap 36 is not engaged with the device 10. In turn, this reduces the likelihood of coring of the septum 22 and fragmentation from shearing forces, while still providing hermetic sealing of the septum 22 and fluid port 12 when the device 10 is not in use.

This configuration may permit needles of relatively large diameter, for example 1 mm diameter, to pass through the septum 22 with a reduced likelihood of coring the septum 22. This configuration may also permit smaller, more delicate needles to be used than in the prior art, since a smaller force is needed to advance the needles through the septum 22, which in turn could allow further miniaturisation of the device 10 or the use of more needles 24 in a single device 10. When attached, the cap 36 also acts to protect the septum 22 from external trauma or damage from ultraviolet light.

The cap 36 may comprise one or more protrusions 33 (not visible in FIG. 4), for example annular ridges. The protrusions 33 may be configured to compress the septum 22, for example by producing focal compression on the septum 22. The protrusions 33 may be configured to produce compression localised at the entrance to the fluid port 12. The protrusions 33 may compress the septum 22 around a region of the septum 22 where the needle 24 is advanced through the septum 22. For example, where the septum 22 is a pre-pierced or split septum, the region may include the piercing or split in the septum 22. The protrusions 33 may compress the septum 22 from a side of the septum 22 that faces the exterior of the housing 14. Where the device 10 comprises plural fluid tubes 20 connected to the fluid port 12, the device 10 may comprise plural protrusions 33 configured to seal each fluid tube 20 individually, and prevent cross-leakage of fluid between the fluid tubes 20. The protrusions 33 may be provided by a removable component of the cap 36, such as the disc 38 in the embodiment of FIG. 4. Where the housing 14 comprises protrusions 25 configured to compress the septum 22 as described above, the protrusions 25 of the housing 14 and the protrusions 33 of the cap 36 may have a matching shape and/or size such that they interact to compress the septum 22 between the two sets of protrusions 25, 33. The removable component may be manufactured of metal or plastic, such as polytetrafluoroethylene (PTFE). This configuration may simplify manufacturing, by allowing that only the removable component needs to be changed for different configurations of the device 10 having different shapes and/or numbers of fluid tubes 20. The septum-contacting surface within the cap 36 may have antibacterial properties, for example via silver impregnation.

The cap 36 may be configured to provide a seal around the septum 22, for example by sealing around the perimeter of the septum. The cap 36 may comprise a sealing member 19 (not visible in FIG. 4, but visible in FIG. 9) to provide the (preferably hermetic) seal between the cap 36 and the extracorporeal portion 16 of the housing 14. The sealing member 19 may be a compliant silicone or polyurethane ring attached to the underside of the cap 36. The sealing member 19 may be configured to engage with a proximal surface of the extracorporeal portion 16 of the housing 14. When the cap 36 is engaged with the extracorporeal portion 16, the sealing member 19 is compressed between the extracorporeal portion 16 of the housing 14 and the cap 36, creating a (preferably hermetic) seal around the septum 22. The seal around the septum 22 seals (preferably hermetically) a region or volume around the septum 22, thereby preventing fluid or gas access to the septum 22 from the exterior of the device 10. This further assists in ensuring cleanliness of the surface of the septum 22, and reducing the risk of dirt, debris, or pathogens being introduced into the CNS via the fluid port 12. As with the septum-contacting surface within the cap 36, the sealing member 19 may have antibacterial properties, for example via silver impregnation.

When the device 10 is used to deliver fluid to, or extract fluid from, the CNS, the cap 36 is removed. Preferably, the septum 22 is cleaned with an antiseptic solution, and a needle 36 is inserted through the septum 22 and into communication with the fluid port 12 to transfer fluid. During this transient exchange while the cap 36 is removed, the septum 22 need only exert sufficient pressure to maintain a fluid and air seal to counteract the intracranial pressure. The intracranial pressure is generally 7-15 mmHg, rising to a maximum of 25 mm Hg, so a relatively thin and compliant septum 22 is still sufficient to maintain a hermetic seal. Depending on the desired application, the septum 22 may be chosen to be sufficiently pliable that a small amount of fluid leaks out from the fluid port 12 while the septum 22 is exposed with no cap 36 or needle 24 engaged. This may be advantageous, because a small outward flow of liquid further reduces the likelihood of unwanted material passing into the CNS through the fluid port 12.

The cap 36 may be configured to engage with the extracorporeal portion 16 using a mechanical connection. The mechanical connection may comprise one or more of a thread, a snap fit connection, an interference fit connection, and a grub screw. Where a thread is used, the thread may be a single entry thread, or a thread having a plurality of entries. In FIG. 4, the mechanical connection comprises a double-entry thread 40. An example of a suitable thread is a Spiralock thread provided by Spiralock Corp. The mechanical connection may be tamper-proof, for example requiring a specialised or unique tool to apply or remove the cap 36. The mechanical connection may comprise a first connection feature on the cap 36 and a second connection feature on the extracorporeal portion 16. The cap 36 may be configured to engage with the extracorporeal portion 16 by engagement of the first connection feature with the second connection feature. In the embodiment of FIG. 4, the second connection feature is provided by the male double-entry thread 40. The first connection feature is provided by corresponding female threads in the cap 36.

The mechanical connection may be configured such that a predetermined compression force is applied to the septum 22 when the cap 36 is engaged with the extracorporeal portion 16. The predetermined force may be sufficient to compress the septum 22 to provide a hermetic seal, without being so large as to risk causing damage to the septum 22.

The mechanical connection may be configured to provide an indication of when the mechanical connection is applying the predetermined force, i.e. when the mechanical connection is fully engaged with the extracorporeal portion 16. This could be using a visual marking or a tactile feedback when the mechanical connection reaches a predetermined position. Additionally or alternatively, the mechanical connection may be configured such that it cannot apply a force greater than the predetermined force, for example by having a physical limit on how tightly it can be engaged.

The mechanical connection may be configured to reversibly lock the cap 36, preferably at a position at which the predetermined force is applied. This can prevent the cap 36 from loosening while the device 10 is not in use and ensure consistent application of the predetermined force. The reversible locking may also contribute to providing a tactile feedback for when the cap 36 is fully engaged with the extracorporeal portion 16, as described above. When combined with the feature of being tamper-proof, the reversible locking may also reduce the risk of patient tampering with the cap 36 and/or device 10.

The cap 36 may comprise tool-engagement features that allow a tool to engage with the cap 36 to tighten or loosen the cap 36. In FIG. 4, the tool-engagement features comprise radial grooves 42, into which a specialised screwdriver head can lock. The specialised nature of the tool-engagement features may contribute to the tamper-proof characteristics of the cap 36 and mechanical connection. Tool-engagement features also permit the use of tools to provide the advantage of easy handling of the small components of the device 10, and reduce direct contact between the user and the components of the device 10, which reduces the risk of bacterial contamination.

FIG. 5 shows an embodiment in which the device 10 comprises a connector cap 44 configured to engage with the extracorporeal portion 16 of the housing 14. The connector cap 44 may comprise a needle 24 configured to make fluid connection with the fluid tube 20 via the fluid port 12. The needle 24 is a hollow needle through which fluid can pass. The connector cap 44 has a similar outer shape and size to the cap 36. This may be convenient in some situations, but is not essential. Preferably, where the extracorporeal portion 16 and/or the connector cap 44 are substantially cylindrical, the needle 24 is located co-axially in the connector cap 44. This aids in the correct positioning of the needle 24, because it reduces the constraints on the angular position of the connector cap 44 relative to the extracorporeal portion 16.

The connector cap 44 may be configured to engage with the extracorporeal portion 16 using a mechanical connection. The mechanical connection may comprise one or more of a thread, a snap fit connection, an interference fit connection, and a grub screw. Where a thread is used, the thread may be a single entry thread, or a thread having a plurality of entries. The mechanical connection may be tamper-proof as described above for the cap 36. The mechanical connection may comprise a first connection feature on the connector cap 44 and a second connection feature on the extracorporeal portion 16. The connector cap 44 may be configured to engage with the extracorporeal portion 16 by engagement of the first connection feature with the second connection feature. In the embodiment of FIG. 5, the second connection feature is provided by the male double-entry thread 40. The first connection feature is provided by corresponding female threads 41 in the connector cap 44.

The mechanical connection may be configured such that, upon engagement of the connector cap 44 with the extracorporeal portion 16, the needle 24 advances a predetermined distance through the septum 22. The predetermined distance may be sufficiently large to allow the needle 24 to make fluid connection with the fluid tube 20 via the fluid port 12, without being so large as to risk causing damage to the fluid port 12, fluid tube 20, or the needle 24 by forcing these components together in an unintended manner.

The mechanical connection may be configured to provide an indication of when the needle 24 is advanced by the predetermined distance, i.e. when the mechanical connection is fully engaged with the extracorporeal portion 16. This could be using a visual marking or a tactile feedback when the mechanical connection reaches a predetermined position. Additionally or alternatively, the mechanical connection may be configured such that the needle 24 cannot advance further than the predetermined distance, for example by having a physical limit on how far the needle 24 can be advanced.

The mechanical connection may be configured to reversibly lock the connector cap 44, preferably at a position at which the needle 24 is advanced by the predetermined distance through the septum 22. The reversible locking may also contribute to providing a tactile feedback for when the connector cap 44 is fully engaged with the extracorporeal portion 16. The reversible locking may be achieved with a lockable screw thread or similar.

The connector cap 44 may be configured to compress the septum 22 when the connector cap 44 is engaged with the extracorporeal portion 16 of the housing 14. The connector cap 44 may compress the septum 22 by applying a force perpendicular to a plane of the septum 22, i.e. parallel to a direction in which the needle 24 of the connector cap 44 is intended to be inserted through the septum 22, similarly as described for the cap 36 above. To achieve this, the connector cap 44 may comprise one or more protrusions configured to compress the septum 22 around the needle 24 once the needle 24 has been advanced by the predetermined distance. The protrusion may be in the form of an annular ridge around the needle 24. The protrusions may be configured to compress the septum 22 by producing focal compression on the septum 22. The protrusions may be configured to produce compression localised at the entrance to the fluid port 12. The protrusions may compress the septum 22 around a region of the septum 22 where the needle 24 is advanced through the septum 22. For example, where the septum 22 is a pre-pierced or split septum, the region may include the piercing or split in the septum 22. The protrusions may compress the septum 22 from a side of the septum 22 that faces the exterior of the housing 14.

Fluid from the needle 24 may be supplied at higher pressure than the typical intracranial pressure during infusions, in order to force the fluid into the CNS. Consequently, the septum 22 may not ordinarily provide sufficient sealing force around the needle to prevent leakage of fluid. This may be especially the case where a relatively thin and compliant septum 22 is used in the device 10 intended for use with the cap 36. Providing protrusions in the connector cap 44 to compress the septum 22 around the needle 24 means that when fluid is introduced via the second fluid tube 46 and needle 24, there will be no leakage of fluid to the atmosphere. Where the housing 14 comprises protrusions 25 configured to compress the septum 22 around the region where the needle 24 will be advanced through the septum 22, the protrusions 25 of the housing 14 and the protrusions of the connector cap 44 may have a matching shape and/or size such that they interact to further compress the septum 22 between the two sets of protrusions.

The connector cap 44 may comprise tool-engagement features that allow a tool to engage with the connector cap 44 to tighten or loosen the connector cap 44, substantially as described for the cap 36. In FIG. 5, the tool-engagement features comprise radial grooves 42, into which a specialised screwdriver head can lock.

As shown in FIG. 5, the connector cap 44 may further comprise a second fluid tube 46 in fluid connection with the needle 24 and extending from a side of the connector cap 44 opposite to the needle 24. The second fluid tube 46 provides an extracorporeal extension tube for connecting the device 10 to a source (or drain) of fluid to (or from) the CNS via the fluid port 12 and fluid tube 20.

The connector cap 44 may further comprise a plurality of grooves 42 configured to retain the second fluid tube 46, as shown in the inset to FIG. 5. The plurality of grooves 42 preferably comprises four grooves, more preferably six grooves. The second fluid tube 46 is preferably flexible (similar to the fluid tube 20), and the grooves 42 may be configured to retain the second fluid tube 46 by press-fitting of the second fluid tube 46 into one of the grooves 42. The grooves 42 may be substantially horizontal. The grooves 42 permit the second fluid tube 46 to enter the connector cap 44 through the bottom substantially vertically but exit the connector cap 44 in a sideways direction. The configuration of the connector cap 44 and grooves 42 may allow the second fluid tube 46 to be bent through a controlled radius to prevent kinking of the second fluid tube 46, and to retain the second fluid tube 46 along a desired radial trajectory for connection to a source (or drain) of fluid. This configuration provides a small and low profile means of connection to a pump, for example an ambulatory pump for chronic drug administration. In the example of FIG. 5, the grooves 42 also function as tool-engagement features, as mentioned above in relation to the cap 36.

The device 10 comprising the connector cap 44 may be provided as a part of a kit for administration of fluid input or removal comprising a specialised screwdriver for facilitating engagement of the connector cap 44 with the extracorporeal portion 16. Where the connector cap 44 comprises grooves 42, the specialised screwdriver preferably has teeth at radial positions corresponding to those of the grooves 42 in the connector cap 44. The specialised screwdriver is preferably hollow, and comprises one fewer teeth than the number of grooves 42 in the connector cap 44. This allows the specialised screwdriver to engage the connector cap 44 without interfering with the second fluid tube 46 when secured in place inside a groove, since the second fluid tube 46 can extend radially through the opening provided by the missing tooth of the screwdriver. The specialised screwdriver also provides the advantage of easy handling of the small components of the device 10, and reduces direct contact between the user and the components of the device 10, which reduces the risk of bacterial contamination. In practice, the connector cap 44 may be supplied separately from the other parts of the device 10 such as the housing, for example in a kit comprising the connector cap 44 and the screwdriver 60. Supplying the connector cap 44 separately may be suitable where the connector cap 44 is, for example, supplied as a sterile consumable intended for use in a single instance of fluid input or removal.

The connector cap 44 having a single needle 24 placed centrally co-axially with the connector cap 44 and extracorporeal portion 16 is most suitable where the device 10 comprises a single fluid tube 20. Where multiple fluid tubes 20 are present in the device 10, a connector 50 and a separate guide member 48 may be provided, as illustrated in FIG. 6. The use of the guide member 48 and connector 50 is not restricted to use where multiple fluid tubes 20 are present, however, and can be used in a device 10 having only a single fluid tube 20.

FIG. 6 shows an embodiment of the device 10 comprising a guide member 48 and a connector 50 configured to engage with the guide member 48. The guide member 48 and/or the connector 50 may be made from plastic (such as PEEK) or a metal, such as titanium.

The connector 50 may comprise one or more needles 24 configured to make fluid connection with respective fluid tubes 20 via the fluid port 12. In FIG. 6, the connector 50 comprises four needles 24, and the device 10 comprises four fluid tubes 20 (not shown in FIG. 6, but located inside the extracorporeal portion 16). Similarly to the connector cap 44 described above, the connector 50 may comprise one or more second fluid tubes 46 in fluid connection with respective ones of the needles 24, which allow for the needles 24 to be connected to a source (or drain) of fluid. For example, the connector 50 shown in FIG. 6 comprises four second fluid tubes 46, one in fluid connection with each of the four needles 24.

Where the device 10 comprises a septum 22 sealing the fluid port 12, the connector 50 and the guide member 48 may be configured such that, upon engagement of the connector 50 with the guide member 48, each of the needles 24 advances a predetermined distance through the septum 22. The predetermined distance may be sufficiently large to allow the needles 24 to make fluid connection with the fluid tubes 20 via the fluid port 12, without being so large as to risk causing damage to the fluid port 12, fluid tubes 20, or the needles 24 by forcing these components together in an unintended manner.

The connector 50 may engage with the guide member 48 via a mechanical connection, for example one or more of a thread, a snap fit connection, an interference fit connection, and a grub screw. Where a thread is used, the thread may be a single entry thread, or a thread having a plurality of entries. The mechanical connection may be configured to provide an indication of when the needles 24 are advanced by the predetermined distance, i.e. when the mechanical connection is fully engaged with the extracorporeal portion 16. This could be using a visual marking or a tactile feedback when the mechanical connection reaches a predetermined position. Additionally or alternatively, the mechanical connection may be configured such that the needles 24 cannot advance further than the predetermined distance, for example by having a physical limit on how far the needles 24 can be advanced. The mechanical connection may be configured to reversibly lock the connector 50, preferably at a position at which the needle 24 is advanced by the predetermined distance through the septum 22. The reversible locking may also contribute to providing a tactile feedback for when the connector 50 is fully engaged with the guide member 48.

The connector 50 may comprise one or more protrusions to compress the septum 22 around the needles 24 once the needles have been advanced by the predetermined distance. The protrusions may be in the form of annular ridges around each of the needles 24. Fluid from the needles 24 may be supplied at higher pressure than the typical intracranial pressure during infusions, in order to force the fluid into the CNS. Consequently, the septum 22 may not ordinarily provide sufficient sealing force around the needle to prevent leakage of fluid. This may be especially the case where a relatively thin and compliant septum 22 is used in the device 10 intended for use with the cap 36. Providing protrusions in the connector 50 to compress the septum 22 around the needles 24 means that when fluid is introduced via the second fluid tubes 46 and needles 24, there will be no leakage of fluid to the atmosphere, or cross-contamination of fluid between the needles 24 and fluid tubes 20.

The guide member 48 may be formed integrally with the extracorporeal portion 16 of the housing 14. Alternatively, as shown in FIG. 6, the guide member 48 may be removably attached to the extracorporeal portion 16 of the housing 14, for example using a mechanical connection. The mechanical connection may comprise one or more of a thread, a snap fit connection, an interference fit connection, and a grub screw. Where a thread is used, the thread may be a single entry thread, or a thread having a plurality of entries. For example, in FIG. 6, the mechanical connection comprises a grub screw 52 in the guide member 48 configured to engage with an outer surface of the extracorporeal portion 16. The mechanical connection may comprise a first connection feature on the guide member 48 and a second connection feature on the extracorporeal portion 16, such that the guide member 48 is removably attached to the extracorporeal portion 16 by engagement of the first connection feature with the second connection feature. In FIG. 6, the mechanical connection comprises first and second connection features in addition to the grub screw 52. The second connection feature may be provided by three hemispherical protrusions 56 on the extracorporeal portion 16, while the first connection feature may be provided by two conical recesses in the underside of the guide member 48 (not visible in FIG. 6), and one conical recess in the distal end of the grub screw 52. Tightening of the grub screw drives all 3 conical recesses in the guide member 48 and grub screw 52 onto the three protrusions 56 on the extracorporeal portion 16 in a unique and repeatable orientation.

When the connector 50 comprises plural needles 24 and the device 10 comprises plural fluid tubes 20, the axis of the connector 50 must be correctly aligned with the axis of the housing 14 to ensure that each needle 24 makes fluid connection with the correct corresponding fluid tube 20 via the fluid port 12. This is necessary to ensure that the correct fluids can be delivered to (or removed from) the correct regions of the CNS. To achieve this, the connector 50 and the guide member 48 are configured such that, upon engagement of the connector 50 with the guide member 48, each of the needles 24 adopts a predetermined position relative to the respective one of the fluid tubes 20.

The guide member 48 may take a variety of forms in order to achieve the correct relative alignment. For example, the guide member 48 may comprise a plurality of guide posts 58. The connector 50 may then be configured to engage with the guide posts 58. The use of guide posts 58 reduces the angular deviation possible when engaging the connector 50 compared to prior art designs where a relatively short, wide cylinder is engaged with a recess in the skull. This is particularly true when the guide posts 58 are designed to have a large aspect ratio of length to diameter. The ratio of the length of the guide post 58 to its diameter may be at least 2:1, preferably at least 3:1.

In FIG. 6, the connector 50 comprises a cam 54 configured to engage with the guide posts 58 present on the guide member 48 (two guide posts 58 in the case of FIG. 6). The cam 54 and guide posts 58 are configured such that rotation of the cam 54 causes the advancement of the needle 24 by the predetermined distance to the predetermined position. This process is shown in further detail in FIG. 7 and FIG. 8.

In FIG. 7, the guide member 48 has been attached to the extracorporeal portion 16 by engagement of the first and second connection features and tightening of the grub screw 52 using screwdriver 60. The connector 50 can then be engaged with the guide member 48. In FIG. 8, the connector 50 is in the process of being engaged with the guide member 48. The cam 54 is engaged with the guide posts 58, and the interaction of ramped surfaces within the cam 54 with corresponding features at the ends of the guide posts 58 as the cam 54 rotates causes the connector 50 to become fully engaged with the guide member 48, such that the needles 24 are advanced longitudinally by the predetermined distance. Rotation of the cam 54 relative to the rest of the connector 50 may be achieved manually by a user gripping the cam 54, or using a tool or actuator. The cam 54 and guide posts 58 may be further configured to reversibly lock the needles 24 at the predetermined position once the needles 24 have advanced by the predetermined distance.

Following fluid transfer, the connector 50 may be disengaged from the guide member 48 by rotating the cam 54 to unlock it, and then withdrawing the needle(s) 24 through the septum 22. During this manoeuvre, any protrusions that may be present in the connector 50 first disengage from the septum 22 as the cam 54 is unlocked, reducing the compressive force on the septum 22. The needle 24 can then be removed with less shear forces imposed on the septum 22, reducing wear on the septum 22.

Where the guide member 48 is removably attached to the extracorporeal portion 16, it is similarly important that the guide member 48 be correctly aligned with the extracorporeal portion 16 to ensure that the needles 24 of the connector 50 make fluid connection with the correct ones of the fluid tubes 20. Therefore, the mechanical connection used to attach the guide member 48 to the extracorporeal portion 16 may be configured such that, upon attachment of the guide member 48 to the extracorporeal portion 16, the guide member 48 adopts a predetermined position relative to the extracorporeal portion 16.

The device 10 including the guide member 48 and connector 50 may be provided as part of a kit 62 for administration of fluid input or removal such as shown in FIG. 16. The kit 62 may further comprise a screwdriver 60 for facilitating engagement of the connector 50 with the guide member 48 and/or the attachment of the guide member 48 to the extracorporeal portion 16. The kit 62 may further comprise extension lines 64, which can be connected to the second fluid lines 46 to facilitate connection to a source or drain of fluid located at greater distances from the patient. In practice, the guide member 48 and connector 50 may be supplied separately from the other components of the device 10 such as the housing 14. Supplying the guide member 48 and connector 50 separately may be suitable where the connector 50 is, for example, supplied as a sterile consumable intended for use in a single instance of fluid input or removal.

FIGS. 9-13 show another embodiment of the device 10 comprising a cap 36, where the device 10 may comprise two fluid tubes 20. This embodiment may be particularly suited for intrathecal delivery of therapeutic agents, having two fluid tubes 20 with a larger diameter. The embodiment shown in FIGS. 9-13 is substantially similar to the embodiments discussed above, but having a different, non-cylindrical shape of the extracorporeal portion 16 and cap 36. In this embodiment, the extracorporeal portion 16 may comprise a substantially flat and straight portion, and a curved, U-shaped portion. The straight edge and curved edge appear straight and curved respectively when viewed from above. The non-circular shape allows an engaging cap 36 or guide member 48 to be appropriately rotationally positioned when engaged with the extracorporeal portion. Features of the embodiments discussed above may be combined with or used in the embodiment of FIGS. 9-13 as appropriate.

In FIG. 9, the protrusions 33 on the cap 36 are visible. Since the embodiment of FIGS. 9-13 comprises plural fluid tubes 20, the cap 36 may comprise plural protrusions 33 configured to seal each fluid tube 20 individually, and prevent cross-leakage of fluid between the fluid tubes 20.

In the embodiment of FIG. 9, the mechanical connection by which the cap 36 engages with the extracorporeal portion 16 may comprise a grub screw 52 in the cap 36, and first and second connection features. The first connection feature may be provided by hemispherical protrusions 56 on the cap 36. The second connection feature may be provided by recesses 31 in the outer surface of the extracorporeal portion 16. The hemispherical protrusions 56 and grub screw 52 may be configured to engage with the recesses 31 to secure the cap 36 to the extracorporeal portion 16. The hemispherical shape of the protrusions 56 may be advantageous in allowing the protrusions 56 to act as a fulcrum to rotate the cap 36 into the closed position. This is also facilitated by two or more of the first connection features (in this case the two hemispherical protrusions 56) being located in a first plane and the corresponding two or more second connection features (in this case two of the recesses 31) being located in a second plane.

FIG. 10 shows exploded views of the device 10 with cap 36 at various levels of disassembly. The fluid port 12 may comprise a funnel portion 70 fitted to the proximal end of each fluid tube 20, to help guide needles 24 into fluid connection with the respective fluid tube 20. The funnel portions 70 may be fitted to the respective fluid tubes 20 via a bayonet fitting, but any suitable, fluid-tight fitting may be used in general. After fitting the funnel portions 70 to the fluid tubes 20, the funnel portions 70 with the fluid tubes 20 attached may be press-fitted into channels in the housing 14. The channels may be provided by profiled holes in the housing 14. Where the housing 14 comprises a moulded portion 26, the channels may be at least partially provided by the moulded portion 26.

The channels and funnel portions 70 may be configured such that, when the funnel portions 70 are fully seated in the channels, the rims of the funnel portions 70 protrude above an inner flat surface of the extracorporeal portion 16 of the housing 14 that accommodates the septum 22. The protruding rims may then provide the protrusions 25 of the housing 14, which can act as valve seatings for the septum 22 around where the needle 24 penetrates the septum 22. This arrangement has the advantage that no separate components are required within the housing 14 to provide the protrusions 25, which would need to be joined or sealed to the housing 14. Thereby, the manufacturing complexity is reduced.

In the process of press-fitting each of the funnel portion 70 into the channels in the housing 14, each fluid tube 20 is compressed between the bayonet fitting of the funnel portion 70 within the bore of the fluid tube 20 and the wall of the channel. This further secures the fluid tubes 20 to the housing 14 and creates a seal (preferably effective to seal against fluid and gas, preferably a hermetic seal) between the exterior surface of the fluid tube 20 and an interior surface of the housing 14. The funnel portions 70 and corresponding channel thereby provide a simple, combined solution for providing the protrusion 25, sealing the exterior surface of the fluid tube 20 to the interior surface of the housing 14, and securing the fluid tube 20 in place in the housing 14.

FIG. 11 shows a cross-sectional view and a top-down view of the device with the guide member 48 and connector 50 engaged with the extracorporeal portion 16 of the housing 14. In this view, the protrusions 55 of the connector 50 that compress the septum 22 are visible. FIG. 12 shows an exploded perspective view of the device 10 with guide member 48 and connector 50, analogously to FIG. 6. FIG. 13 shows engagement of the connector 50 with the guide member 48, analogously to FIGS. 7 and 8.

FIG. 14 illustrates the positioning of the device 10 of FIGS. 9-13 once implanted and connected to the first catheter 80 and lumbar catheter 82.

The device 10 may be provided as part of a kit for implanting a device 10 for providing fluid access to the central nervous system of a mammal. The kit comprises the device 10 according to any suitable embodiment described above, and a predetermined quantity of an acrylic cement.

As mentioned above, the fluid tube 20 may be configured to run along a trench 32 formed in the outermost surface 30 of the skull. Once the fluid tube 20 is placed within the trench 32, the trench is preferably filled with an acrylic cement. However, acrylic cement is typically supplied for orthopaedic procedures in quantities much larger than required to fill the trench 32. This can lead to significant wastage of acrylic cement, due to its limited working time once a package is opened. Therefore, supplying a kit comprising the device 10 and a suitable quantity of acrylic cement for filling the trench can significantly reduce wastage of acrylic cement and the associated cost. The kit also makes the procedure of implanting the device 10 faster and more convenient, due to the easy availability of a suitable quantity of acrylic cement. This removes the need for opening of a larger package of acrylic cement and measuring of an appropriate quantity thereof during the surgical procedure. Measuring of the acrylic cement during surgery would otherwise be necessary, since the cement could not be pre-measured significantly prior to the procedure due to its relatively short working time. Preferably, the acrylic cement comprises an antimicrobial agent. This reduces the chance of infection following implantation of the device 10.

The acrylic cement could be included with the device 10 in a kit such as the one shown in FIG. 16, which also contains other components of the device 10 such as the guide member 48 and connector 50. The kit may further comprise a cap 36. The kit may also include a tool such as the screwdriver 60 to reversibly secure the guide member 48 or the cap 26 to the extracorporeal portion 16 of the housing 14 of the device 10. The kit may further include a spatula or similar tool suitable for spreading the acrylic cement and removing excess cement. The kit may further include fittings used to connect the fluid tubes 20 to a catheter or cannula for delivery or removal of fluids from the CNS. Such fittings may comprise bayonet fittings. The fittings may be marked with unique identifiers (for example a numeral or coloured band) to distinguish them from one another and to aid in ensuring that the correct fluid tube 20 is connected to the correct catheter or cannula.

A method of assembly of the device 10 may be provided as follows. The method may comprise inserting a proximal end of the fluid tubes 20 into the housing 14 through the lowermost surface 18. Where the device 10 comprises one or more funnel portions as described in relation to FIG. 10, the method may comprise a step of fitting the funnel portion(s) 70 to respective fluid tube(s) 20, for example by fitting a bayonet fitting of the funnel portion 70 inside the fluid tube 20 to create an interference fit. The method may further comprise fitting the funnel portion(s) 70 into respective channel(s) in the housing 14. Preferably, the fitting of the funnel portions 70 into the channels is such that the wall of each fluid tube 20 is compressed between the funnel portion 70 and the channel. This serves to create a strong permanent connection. Fitting the funnel portions 70 into the channels may comprise press-fitting the funnel portions 70 into the channels.

Where the device 10 comprises a moulded portion 26, the method may comprise a step of fitting the moulded portion 26 into the extracorporeal portion 16 of the housing 14. Where the moulded portion 26 provides the channels for the funnel portions 70, the moulded portion 26 may be fitted into the extracorporeal portion 16 before or after fitting the funnel portions 70 into the channels.

The method may comprise fitting the septum 22 into the extracorporeal portion 16 of the housing 14. This step may be carried out before or after fitting the moulded portion 26 into the extracorporeal portion 16, depending on the design of the housing 14. For example, in the device 10 of FIG. 4, the septum 22 is fitted into the housing 14 before or simultaneously with the moulded portion 26. However in the device of FIG. 10, the septum 22 may be fitted after the moulded portion 26, if a moulded portion 26 is present. The method may comprise, after the septum 22 and moulded portion 26 are fitted, inserting a retaining member 23 into the housing 14. The inserting of the retaining member 23 may compress the septum 22 around an edge of the septum 22.

Any of the devices 10 discussed above can be implanted using a method of implanting a device for providing fluid access to the central nervous system of a mammal. FIG. 17 shows a flowchart of the method, and FIG. 18 illustrates visually some steps of the method. The device 10 is implanted on the skull at an implantation site. Preferably, the implantation site is on the posterior auricular temporal bone or on the parietal bone. The method of implanting the device 10 will generally be performed on an anaesthetised patient.

The method comprises removing at step S10 an area of scalp at the implantation site of sufficient size to accommodate the extracorporeal portion 16 of the housing 14 of the device 10. This is illustrated in FIG. 18a). After cleaning the skin at the selected implantation site with antiseptic solution, the area of scalp is removed. A punch hole may be made through the scalp to the skull surface, for example using a skin biopsy punch of a size that will accommodate the extracorporeal portion 16 of the device 10. Where the extracorporeal portion 16 is substantially cylindrical, this may be a punch similar in diameter to the extracorporeal portion 16, for example a diameter of 2-7 mm, preferably 4 mm or 5 mm. The sharp cylindrical end of the punch will mark the skull surface.

The method comprises removing at step S20 subcutaneous fat and hair follicles in a predetermined area around the implantation site. This is illustrated in FIG. 18b). This may be achieved by inserting an ultrasonic aspirator through the punch-hole. The predetermined area may comprise a substantially circular area having a radius of 0.5-2 cm, preferably approximately 1 cm from the centre of the removed area of scalp (i.e. the punch hole). Removing subcutaneous fat and hair follicles is not essential, but is advantageous in reducing the mobility of the scalp at the interface between the skin and the device 10. This in turn assists tissue adhesion and integration with the device 10.

The method comprises forming at step S30 a trench 32 in the outermost surface of the mammal's skull. The trench 32 may be formed by making a rostro-caudal incision through the scalp and centred on the removed area of scalp. The scalp and periosteum are then retracted. Alternatively a C-shaped incision with a radius of 1-3 cm, preferably approximately 2 cm is made centred on the removed area of scalp, and the scalp with periosteum retracted. From the centre of the removed area of scalp, a radial trench 32 is then made in the skull. The trench may be formed with a burr of 1-4 mm, preferably 2 mm or 3 mm burr. The trench is made of sufficient depth to accommodate the one or more fluid tubes 20 of the device 10 below the outermost surface of the skull. The trench 32 extends from the implantation site towards a cannula providing fluid connection to the central nervous system of the mammal. The trench 32 need not extend all the way to the cannula. Preferably, the trench extends at least 5 mm, preferably at least 10 mm towards the cannula to accommodate the fluid tube 20 that connects to the implanted cannula. The trench 32 does not penetrate the inner surface of the skull. The trench can be made using hand held tools by eye as there is no requirement for an accurately defined shape or pathway. The tool can have a depth limiter to fix the depth of the trench but otherwise provide the surgeon with freedom as to the path the trench takes.

The method comprises connecting at step S40 a fluid tube 20 of the device 10 to the cannula. Optionally, plural cannulas may be present, and the device 10 may comprise plural fluid tubes 20, which are connected to respective ones of the cannulas. This step is illustrated in FIG. 18c). Where the device 10 comprises funnel portions as described in relation to FIG. 10, the method may comprise a step of fitting the funnel portions 70 to the fluid tube 20, and/or fitting the funnel portions 70 into the channels in the housing 14. This step should be carried out before the attaching of the device to the outermost surface 30 of the skull. It may be preferable to carry out this step before the step S40 of connecting the fluid tube 20 to the cannula, as it may be easier to fit the funnel portions 70 to the fluid tube 20 before the fluid tube 20 is connected to the cannula. The fitting of the funnel portion(s) 70 to the fluid tube(s) 20 and/or the fitting of the funnel portion(s) 70 into the channel(s) in the housing 14 is preferably carried out before implantation as part of the assembly of the device 10, as described above, but may be carried out during the implantation in some situations.

The method comprises filling at step S50 the trench 32 with an acrylic cement. The trench 32 is preferably slightly overfilled, to ensure that sufficient cement is present to fill any voids or gaps that could harbour infection.

The method comprises inserting at step S60 the fluid tube 20 (or plural fluid tubes 20 if present) into the trench 32. Inserting S60 the fluid tube 20 may comprise bending the fluid tube 20 to run along the trench 32, for example by 90°. The fluid tube or tubes may be applied directly to a cement-filled trench, and may displace cement out of the trench during insertion. The acrylic cement immobilises the fluid tube 20, and fills the space in the skull around the fluid tube 20 to prevent infection. Excess cement may be removed from the skull surface and made level with the skull surface over the trench 32.

Optionally, the step S60 of inserting the fluid tube 20 into the trench 32 may be performed after the step S70 of attaching the device 10. Optionally, the step S50 of filling the trench 32 with an acrylic cement may be performed after the step S60 of inserting the fluid tube 20 into the trench 32 and/or the step S70 of attaching the device 10. However, this is not preferred, because filling the trench 32 with cement after insertion of the fluid tube 20 into the trench 32 increases the likelihood of leaving voids around the fluid tube 20 that could harbour infection.

As the fluid tube 20 is inserted into the trench 32, the fluid tube 20 will displace some of the un-set acrylic cement. This in turn will force the cement beneath the lowermost surface 18 of the device 10, for example after or during the attaching S70 of the device 10. Under compression, the cement will be driven into the interstices in the skull surface and into any spaces between the lowermost surface 18 of the housing 14 and the outermost surface 30 of the skull. This will assist in fixation of the device 10 to the skull and in providing a hermetic seal at the interface between the device 10 and the skull. This also ensures that any spaces or voids around the lowermost surface 18 of the device 10 and the fluid tube 20 are filled with cement to prevent leaving voids that could harbour infection.

The method comprises engaging at step S70 the lowermost surface of the device 10 with the outermost surface 30 of the skull at the implantation site, so as to attach the device to the skull. This is illustrated in FIG. 18d). Engaging the lowermost surface of the device 10 may comprise installing one or more screws 28 into the skull. Engaging the lowermost surface 18 of the device 10 may also comprise impacting the lowermost surface 18 into the outermost surface 30 of the skull, for example using a hollow cylinder placed over the extracorporeal portion 16 of the housing 14 and engaging with the subcutaneous portion 17. This is particularly advantageous where the lowermost surface 18 of the device 10 comprises a plurality of teeth 34. Impacting the lowermost surface 18 into the outermost surface of the skull in this manner will drive the teeth 34 into the outermost surface 30, thereby improving engagement of the device 10 with the skull. This step may also involve using acrylic cement at the join between the lowermost surface 18 of the housing and the outermost surface 30 of the skull. This provides additional adhesive and sealing qualities.

The method comprises a step S80 of closing the wound formed in step S30 of forming the trench 32. This is illustrated in FIG. 18e) If a C-shaped flap was made in step S30 with a punch hole at its centre, then as the flap is turned back the extracorporeal portion 16 of the device 10 is pushed through the hole in the flap prior to closing the periosteum and scalp with sutures. If a linear incision was made in step S30 centred on the removed area of scalp, then the periosteum and scalp are closed on each side of the punch hole.

1. A device for providing fluid access to the central nervous system of a mammal, said device comprising:
a fluid port allowing the delivery or removal of fluid from the central nervous system;
a housing comprising an extracorporeal portion allowing access to said fluid port, and a lowermost surface configured to engage with an outermost surface of the skull; and
a fluid tube connected to the fluid port and extending below the housing through the lowermost surface, wherein:
no part of the housing extends below the lowermost surface; and
the fluid tube is configured to bend so as to run along a trench formed in the outermost surface of the skull.

2. The device of claim 1, wherein no part of the device extends through an interior surface of the skull when the lowermost surface of the housing is engaged with the outermost surface of the skull.

3. The device of claim 1, wherein the lowermost surface is configured to engage with, and attach to, the outermost surface of the skull using a plurality of screws.

4. The device of claim 1, wherein the fluid tube is configured to bend at a point below the lowermost surface.

5. (canceled)

6. A device for providing fluid access to the central nervous system of a mammal, said device comprising:
a fluid port allowing the delivery or removal of fluid from the central nervous system;
a housing comprising an extracorporeal portion allowing access to said fluid port, and a lowermost surface configured to engage with an outermost surface of the skull; and
a fluid tube connected to the fluid port and extending from the housing, wherein:
the lowermost surface of the housing comprises a plurality of teeth for engagement with the outermost surface of the skull.

7. The device of claim 6, wherein the plurality of teeth is distributed over the lowermost surface, for example over at least 50% of an area of the lowermost surface.

8.-9. (canceled)

10. A device for providing fluid access to the central nervous system of a mammal, said device comprising:
a fluid port allowing the delivery or removal of fluid from the central nervous system;
a housing comprising an extracorporeal portion allowing access to said fluid port;
a fluid tube connected to the fluid port and extending from the housing;
a septum sealing the fluid port; and
a cap configured to engage with the extracorporeal portion of the housing and compress the septum when the cap is attached.

11. The device of claim 10, wherein the cap is one or both of:
i) configured to compress the septum by applying a force perpendicular to a plane of the septum; and
ii) configured to provide a seal around the septum.

12. The device of claim 10, wherein the cap is configured to engage with the extracorporeal portion using a mechanical connection, for example one or more of a thread, a snap fit connection, an interference fit connection, and a grub screw.

13. The device of claim 12, wherein the mechanical connection comprises a first connection feature on the cap and a second connection feature on the extracorporeal portion, and the cap is configured to engage with the extracorporeal portion by engagement of the first connection feature with the second connection feature.

14. The device of claim 12, wherein the mechanical connection is configured such that a predetermined compression force is applied to the septum when the cap is engaged with the extracorporeal portion.

15. (canceled)

16. The device of claim 1, further comprising a connector cap configured to engage with the extracorporeal portion of the housing, the connector cap comprising a needle configured to make fluid connection with the fluid tube via the fluid port.

17. The device of claim 16, wherein the connector cap is configured to engage with the extracorporeal portion using a mechanical connection, for example one or more of a thread, a snap fit connection, an interference fit connection, and a grub screw.

18. (canceled)

19. The device of claim 17, wherein the device comprises a septum sealing the fluid port, and the mechanical connection is configured such that, upon engagement of the connector cap with the extracorporeal portion, the needle advances a predetermined distance through the septum.

20. The device of claim 16, wherein the device comprises a septum sealing the fluid port, and the connector cap is configured to compress the septum by applying a force perpendicular to a plane of the septum when the connector cap is engaged with the extracorporeal portion of the housing.

21. The device of claim 16, wherein the connector cap further comprises:
a second fluid tube in fluid connection with the needle and extending from a side of the connector cap opposite to the needle; and
a plurality of grooves configured to retain the second fluid tube.

22. (canceled)

23. A device for providing fluid access to the central nervous system of a mammal, said device comprising:
a fluid port allowing the delivery or removal of fluid from the central nervous system;
a housing comprising an extracorporeal portion allowing access to said fluid port;
a guide member;
one or more fluid tubes connected to the fluid port and extending from the housing; and
a connector configured to engage with the guide member, the connector comprising one or more needles configured to make fluid connection with respective ones of the fluid tubes via the fluid port,
wherein the connector and the guide member are configured such that, upon engagement of the connector with the guide member, each of the needles adopts a predetermined position relative to the respective one of the fluid tubes.

24. The device of claim 23, wherein:
the device comprises a septum sealing the fluid port; and
the connector and the guide member are configured such that, upon engagement of the connector with the guide member, each of the needles advances a predetermined distance through the septum.

25. The device of claim 24, wherein:
the guide member comprises a plurality of guide posts;
the connector comprises a cam configured to engage with the guide posts; and
the cam and guide posts are configured such that rotation of the cam causes the advancement of the needles by the predetermined distance to the predetermined position.

26. The device of claim 25, wherein the cam and guide posts are further configured to reversibly lock the needles at the predetermined position once the needles have advanced by the predetermined distance.

27. The device of claim 23, wherein the guide member is removably attached to the extracorporeal portion of the housing using a mechanical connection, for example one or more of a thread, a snap fit connection, an interference fit connection, and a grub screw.

28.-29. (canceled)

30. The device of claim 1, wherein one or more of:
i) a surface of the housing configured to be in contact with tissue of the mammal has at least one of a texture and coating configured to promote tissue integration;
ii) the housing of the device comprises titanium and/or polyetheretherketone;
iii) the device further comprises a septum sealing the fluid port, wherein the septum is a pre-pierced or split septum; and
iv) the device comprises a septum sealing the fluid port, and the housing comprises one or more protrusions configured to compress the septum.

31.-33. (canceled)

34. A kit for implanting a device for providing fluid access to the central nervous system of a mammal, the kit comprising:
the device of claim 1; and
a predetermined quantity of an acrylic cement.

35. The kit of claim 34, wherein the acrylic cement comprises an antimicrobial agent.

36. A method of implanting a device for providing fluid access to the central nervous system of a mammal, the method comprising:
forming a trench in the outermost surface of the mammal's skull, said trench extending from an implantation site towards a cannula providing fluid connection to the central nervous system of the mammal, wherein the trench does not penetrate the inner surface of the skull;
connecting a fluid tube of the device to the cannula;
filling the trench with an acrylic cement;
inserting the fluid tube into the trench; and
engaging the lowermost surface of the device with the outermost surface of the skull at the implantation site.

37. The method of claim 36, further comprising removing one or both of i) an area of scalp at the implantation site of sufficient size to accommodate an extracorporeal portion of a housing of the device, and ii) subcutaneous fat and hair follicles in a predetermined area around the implantation site.

38.-39. (canceled)
